(19) United States
(12) Patent Application Publication
(10) Pub. No.: US 2025/0065154 A1
(43) Pub. Date: Feb. 27, 2025

(54) ISSUE RESOLUTION FOR RADIOTHERAPY DEVICES

(71) Applicant: Elekta Limited, Crawley (GB)

(72) Inventors: Paul BOXALL, Crawley (GB); Enrique MUNOZ, Crawley (GB); Shufei SHEN, Crawley (GB); Mark COWARD, Crawley (GB)

(21) Appl. No.: 18/720,736

(22) PCT Filed: Dec. 15, 2021
(86) PCT No.: PCT/EP2021/086021

§ 371 Date: Jun. 17, 2024

(51) Int. Cl. A61N 5/10; G16H 40/20; G16H 40/40; G21K 5/04

(52) U.S. Cl. CPC A61N 5/1075; G16H 40/20; G16H 40/40; G21K 5/04; A61N 2005/1089

(57) ABSTRACT

Disclosed herein is a method and system for identifying an issue with a radiotherapy device and outputting a next step, the radiotherapy device comprising a linear accelerator and being configured to provide therapeutic radiation to a patient, the method comprising: obtaining data relating to an operational state of the radiotherapy device or a component thereof identifying an issue with the radiotherapy device based on the obtained data; and outputting a next step based on the identified issue. There is also disclosed a method for limiting or changing a radiotherapy device functionality.

This disclosure relates to radiotherapy devices and, in particular, to the improved issue resolution of such radiotherapy devices.

### BACKGROUND

Radiotherapy devices are an important tool in modern cancer treatment. Radiotherapy devices are large, complex machines with many moving parts and inter-operating mechanisms. As a result, the installation of radiotherapy devices in user locations is a complicated and highly skilled activity. Furthermore, despite precision engineering and rigorous testing, some component parts of radiotherapy machines may start to degrade over the lifetime of the machine. Radiotherapy devices therefore require servicing and maintenance to ensure they continue to operate in an optimal manner and to ensure the machines meet safety and regulatory requirements.

A typical approach towards resolving issues for radiotherapy devices, for example service issues, is to deploy one or more members of a team of highly skilled field-service engineers to the user locations where the radiotherapy devices are located, regardless of the severity of the issue. This approach is however undesirable for numerous reasons.

First, while awaiting the arrival of the field-service engineer, the machine may need to be fully disabled. Accordingly, the downtime of the radiotherapy device exhibiting the issue may be significant. Prolonged lengths of downtime are of course extremely undesirable.

Second, the engineers must travel large distances to attend to the radiotherapy devices. This is expensive and burdensome on the field-service engineers and it will be appreciated that travelling between device sites is not an effective use of a highly skilled engineer's time.

Third, due to the scattering of the field-service engineers between the various user locations, it is difficult for the field-service engineers to exchange information (both with each other and with product research and development teams). Consequently, if a particular field-service engineer does not have personal prior experience of a certain installation or maintenance issue, for example, it may take that engineer a considerable amount of time to fix the issue. In that situation, again, the downtime of the radiotherapy device exhibiting the issue may be significant.

Another problem with current approaches is a potential lack of consistency applied by engineers on-site. Different engineers may address the same issue in different ways, and, coupled with the above-identified communication issues, this can lead to problems where certain devices are serviced in a manner which is sub-optimal when compared to the servicing of other machines.

The present invention seeks to address these and other disadvantages encountered in the prior art by providing a method of resolving issues in radiotherapy devices.

### SUMMARY

Aspects and features of the present invention are defined in the accompanying claims.

### BRIEF DESCRIPTION OF THE FIGURES

Specific embodiments are now described, by way of example only, with reference to the drawings, in which:

FIG. 1 depicts a radiotherapy device which may be used in systems and methods according to the present disclosure;

FIG. 2 depicts a cross-section through part of a radiotherapy device which may be used in systems and methods according to the present disclosure;

FIG. 3a depicts a method according to the present disclosure;

FIG. 3b depicts a method according to the present disclosure;

FIG. 4a depicts a method according to the present disclosure;

FIG. 4b depicts a method according to the present disclosure;

FIG. 5 depicts a schematic diagram of a system according to the present disclosure;

FIG. 6 depicts a method according to the present disclosure; and

FIG. 7 depicts a method according to the present disclosure

### DETAILED DESCRIPTION

Radiotherapy machines are beginning to be configured to produce and record a large amount of data as they operate; for example, radiotherapy machines are configured to provide sensor readings from a variety of different sensors. These sensors produce data which can be stored in a database. Radiotherapy devices may also be configured to allow remote connection, enabling service engineers to access a wealth of information about any connected machine without having to travel to the site where the machine is located. It is expected that, in many cases, machines may be returned to optimal performance without an engineer ever having to physically interact with the machine. However, there will still be occasions where the fault cannot be fixed remotely, and an engineer must be sent to: inspect the machine; determine the nature of the fault; and perform any maintenance required. If the repair involves replacing a part, further machine downtime is required before the machine can be brought back online.

The present methods involve evaluating the condition and/or performance of a component of radiotherapy equipment during its operation in order to identify and determine issues such as operational issues, for example service issues, preferably remotely. For example, it may be determined whether the component is nearing the end of its operational life and thus whether the component should be replaced or repaired. In particular, the present application relates to diagnosing an issue with a radiotherapy device, categorising the issue, and performing one of a potential variety of next steps based on the diagnosed issue and/or the category of the issue. Such techniques are advantageous as they allow a manufacturer or maintenance service provider to only attend the machine when it is known that a) it is necessary to do so, and b) what type of fault they expect to find on-site prior to arrival. The disclosed techniques allow the operation of the device to be monitored which allows, for example, repair and/or replacement of a component of a radiotherapy device to be scheduled for the next convenient service point, and/or to be repaired by the hospital staff according to guidelines approved by the manufacturer. The disclosed methods help to reduce machine downtime and thereby minimise disruption to the machine's normal operation. The disclosed techniques can also be used to more effectively plan machine downtime for times which are more convenient or cost-effective for the owner of the equipment and/or the patients.

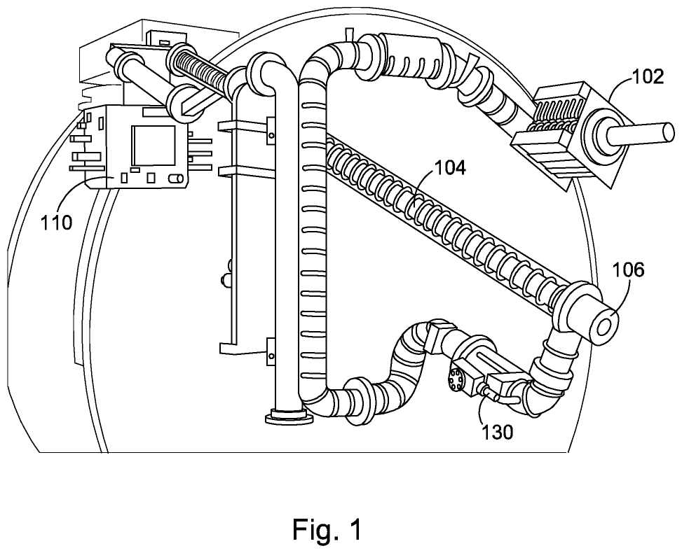

FIG. 1 depicts a linac suitable for delivering, and configured to deliver, a beam of radiation to a patient during radiotherapy treatment. In operation, the linac device produces and shapes a beam of radiation and directs it toward a target region within the patient's body in accordance with a radiotherapy treatment plan.

A medical linac machine is by necessity complex, with many inter-operating component parts. A brief summary of the operation of a typical linac will be given with respect to the linac device depicted in FIG. 1, which comprises a source of radiofrequency waves, a waveguide, a source of electrons, a system capable of creating a strong vacuum comprising one or more vacuum pumps 130, a heavy metal target which produces X-rays when hit by an electron beam, and a complex arrangement of magnets capable of re-directing and focusing the electron beam onto the target. The device depicted in FIG. 1 also comprises a treatment head which houses various apparatus configured to, for example, collimate and shape the resultant X-ray beam.

The source 102 of radiofrequency waves, such as a magnetron, produces radiofrequency waves. The source 102 of radiofrequency waves is coupled to the waveguide 104, and is configured to pulse radiofrequency waves into the waveguide 104. Radiofrequency (RF) waves pass from the source 102 of radiofrequency waves through an RF input window and into a RF input connecting pipe or tube. A source 106 of electrons, such as an electron gun, is coupled to the waveguide 104 and is configured to inject electrons into the waveguide 104. In the source 106 of electrons, electrons are thermionically emitted from a cathode filament as the filament is heated. The temperature of the filament controls the number of electrons injected. The injection of electrons into the waveguide 104 is synchronised with the pumping of the radiofrequency waves into the waveguide 104. The design and operation of the radiofrequency wave source 102, electron source 106 and the waveguide 104 is such that the radiofrequency waves accelerate the electrons to very high energies as they propagate through the waveguide 104.

The design of the waveguide 104 depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide 104. As the electrons are accelerated in the waveguide 104, the electron beam path is controlled by a suitable arrangement of steering magnets, or steering coils, which surround the waveguide 104. The arrangement of steering magnets may comprise, for example, two sets of quadrupole magnets.

Once the electrons have been accelerated, they pass into a flight tube. The flight tube may be connected to the waveguide by a connecting tube. This connecting tube or connecting structure may be called a drift tube. The drift tube also forms part of the vacuum tube. RF waves exit the waveguide via an RF output connecting pipe or tube coupled with the drift tube. RF passes out from the vacuum system via an RF output window which seals the vacuum system.

The flight tube is also kept under vacuum conditions by the pump system. The electrons travel along a slalom path toward the heavy metal target. The target may comprise, for example, tungsten. Whilst the electrons travel through the flight tube, an arrangement of focusing magnets act to direct and focus the beam on the target. The slalom path allows the overall length of the LINAC to be reduced while ensuring that the beam of accelerated electrons, which is comprised of electrons with a small spread of energies, is focused on the target.

To ensure that propagation of the electrons is not impeded as the electron beam travels toward the target, the waveguide 104 is evacuated using a vacuum system comprising a vacuum pump 130 or an arrangement of vacuum pumps. The pump system is capable of producing ultra-high vacuum (UHV) conditions in the waveguide 104 and in the flight tube. The vacuum system also ensures UHV conditions in the electron gun. Electrons can be accelerated to speeds approaching the speed of light in the evacuated waveguide 104. Together, the electron gun 106, waveguide 104 and the flight tube form a vacuum tube in which electrons can be accelerated and directed toward a target in vacuum conditions. In implementations comprising a drift tube connecting the waveguide 104 to the flight tube, the drift tube also forms part of the vacuum tube. The vacuum tube has two ends. The ends may be described as opposing ends. The electron gun 106 is located at a first end of the vacuum tube and the flight tube is located at a second end of the vacuum tube. In other words, the flight tube is located at a distal end of the waveguide 104, and hence vacuum tube, from the electron gun 106.

The combination of the components kept under vacuum, e.g. the vacuum tube and any connecting pipes and tubes, for example those connecting tubes and pipes which couple the RF input and output windows to the vacuum tube and the internal volume of the pumps, may be referred to as the vacuum system. The vacuum system is sealed and is constantly kept under vacuum. To produce the necessary high vacuum conditions, the vacuum system may undergo several stages of pumping before the high-quality vacuum may be maintained using e.g. ion pumps.

When the high energy electrons hit the target, X-rays are produced in a variety of directions. The target is located inside the flight tube, and is located at the end of the flight tube to seal the vacuum system. The flight tube also comprises a target window, which is transparent to X-rays, which is positioned to allow the X-rays which are produced when the linac is in operation to pass from the evacuated flight tube through the target window and into the treatment head 110. At this point, a primary collimator blocks X-rays travelling in certain directions and passes only forward travelling X-rays to produce a cone shaped beam. The X-rays are filtered, and then pass through one or more ion chambers for dose measuring. The beam can be shaped in various ways by beam-shaping apparatus, for example by using a multi-leaf collimator, before it passes into the patient as part of radiotherapy treatment.

In some implementations, the linac is configured to emit either an X-ray beam or an electron particle beam. Such implementations allow the device to provide electron beam therapy, i.e. a type of external beam therapy where electrons, rather than X-rays, are directed toward the target region. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The end of the flight tube may be sealed by a component which comprises both a target and an electron window. It is then possible to swap between the first and second mode by moving the flight tube such that the electron beam points toward either the target or the electron window.

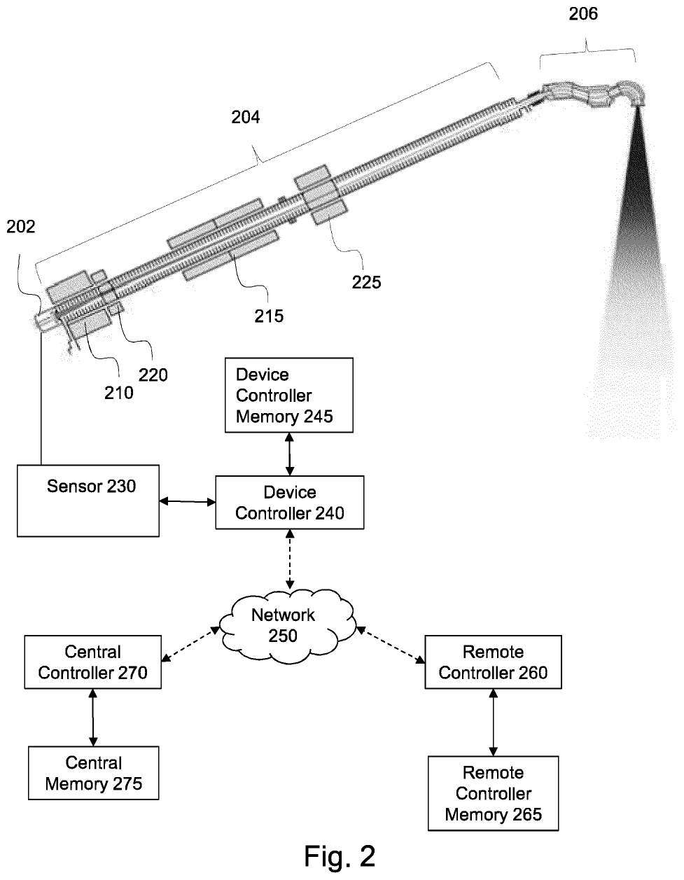

The linac device also comprises several other components and systems. The whole system is cooled by a water cooling system (not shown in the figures). The water cooling system may be used, in particular, to cool the waveguide 104, target, and radiofrequency source 102. In order to ensure the linac does not leak radiation, appropriate shielding is also provided. As will be understood by the person skilled in the art, a linac device used for radiotherapy treatment will have additional apparatus such as a gantry to support and rotate the linac, a patient support surface, and a controller or processor configured to control the linac apparatus. The device will also comprise an imaging apparatus or device such as a KV imager, which may inter-operate with the linac to provide an image-guided radiation therapy system. FIG. 2 depicts a cross-section through part of an exemplary radiotherapy device, and in particular through a linac of a radiotherapy device. As detailed above, the linac may comprise a vacuum tube comprised of an electron gun 202, a waveguide 204, and a flight tube 206. The electron gun 202 is configured to inject electrons into the waveguide 204. In this example, the electron beam may be focused by a first arrangement of focusing magnets 210 and a second arrangement of focusing magnets 215. The beam is ‘steered’, i.e. directed, by a first arrangement of steering magnets 220 and a second arrangement of steering magnets 225. While the linac is in use, the electron gun 202, waveguide 204 and flight tube 206 are kept under high vacuum conditions by a vacuum system or suitable vacuum apparatus.

The radiotherapy device comprises a plurality of sensors. For brevity, only one sensor 230 is depicted in FIG. 2, and reference may be made below to one sensor. However it will be understood that in reality the radiotherapy device may comprise many sensors of differing types, each configured to provide data in the manner described below. The sensors may be any of pressure sensors, current, power or voltage sensors, temperature sensors, etc. The sensor 230 may be comprised within a component of the device, such as within the electron gun 202. The sensor 230 is suitable for detecting, and is configured to detect, a signal associated with the continuing performance of one of the components of the radiotherapy device. For example, the sensor 230 may be configured to provide a signal indicative of the current supplied to the electron gun, i.e. the current which passes through the cathode filament. The sensor 230 is communicatively coupled with a device controller 240, and data relating to the component of the radiotherapy device is communicated to the device controller. The data is measured by the sensor 230 and communicated to the device controller 240 with a particular frequency, for example the sensor 230 may provide a measurement to the device controller 240 every second.

The sensor 230 comprises means with which to communicate with the device controller 240. For example, the sensor may comprise suitable processing circuitry and transmitting antennas. The sensor 240 is electronically and/or communicatively coupled to the device controller 240. The device controller 240 receives signals from the sensor 230 as they are generated, or produced, by the sensor 230. The device controller 240 is electronically and/or communicatively coupled to a device controller memory 245. The device controller 240 and device controller memory 245 may be configured to store signals generated by the current sensor. The generated signals from the sensor comprise sensor data.

The device controller 240 is communicatively coupled to a central controller 270, for example via a network 250. The device controller 240 is configured to transmit, i.e. send, the sensor data to the central controller 270 to be stored on the central controller memory 275. The central memory 275 may comprise a number of different servers as part of a cloud storage solution. The central controller may be communicatively coupled to a plurality of radiotherapy devices via network 250, each of which are configured to transmit signals to the central controller 270 to be stored on central memory 275. Central controller 270 is adapted and configured to process received signals and store them in a database. Processing the signals may comprise, for example, calculating and storing daily averages of particular sensor data.

The radiotherapy device has a variety of sensors, the signals/readings from which are communicated to the device controller 240. The signals may be stored in the device controller memory 245 and/or may be communicated via the network to the central controller 270. The data may be uploaded to the central controller 270 as it is generated, or may be stored on the device controller memory 245 in order to be uploaded as a batch upload, for example at regular time intervals. Alternatively, the data may be continuously gathered by the device controller 240, for example the sensor signals may be sampled every 4 seconds while the device is not delivering radiation, and data is uploaded if the data shows a particular variance from the previously uploaded data point. In a specific implementation, the data points are uploaded when there is a change of +/−0.04, and the device controller looks for a new data item once every 4 seconds while the device is not delivering radiation, and once every second while the linac is delivering radiation.

The data is stored in a database on central memory 275, which may comprise data from sensors, for example the data includes the current values as denoted by signals from the current sensor, the degree of rotation of the gantry, whether or not radiation is being delivered at a particular time and the dose rate and machine output as indicated by a dosimeter or monitor chamber, as well as the water temperature at various points around the water cooling system. These types of data are given to provide examples, and the skilled person will appreciate that a modern linac device may be configured to generate a wealth of data from a large variety of sensors.

The device controller 240 and central controller 270 are both also communicatively coupled to a remote controller 260. The remote controller 260 may access the central database, which stores information and data regarding a plurality of radiotherapy devices, through the database 250 and by using a suitable software platform. The remote controller 260 may also access the device controller 240 to obtain real-time information regarding a particular radiotherapy machine.

The device, central and remote controllers may each be described as a processor, computer, or computing device. The controllers may be connected (e.g., networked) to each other and/or to other machines in a Local Area Network (LAN), an intranet, an extranet, or the Internet. The controllers may each operate in the capacity of a server or a client machine in a client-server network environment, or as a peer machine in a peer-to-peer (or distributed) network environment. The controllers may each be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, where respective controllers are illustrated, the term “controller” shall also be taken to include any collection of machines (e.g., computers) that individually or jointly execute a set (or multiple sets) of instructions to perform any one or more of the methodologies discussed herein.

The approaches of the present disclosure may be embodied on one or more of the device controller memory and the remote controller memory, or any other computer-readable medium. The medium may be a non-transitory computer-readable medium. The computer-readable medium carries computer-readable instructions arranged for execution upon a processor so as to make the controller/processor carry out any or all of the methods described herein. The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

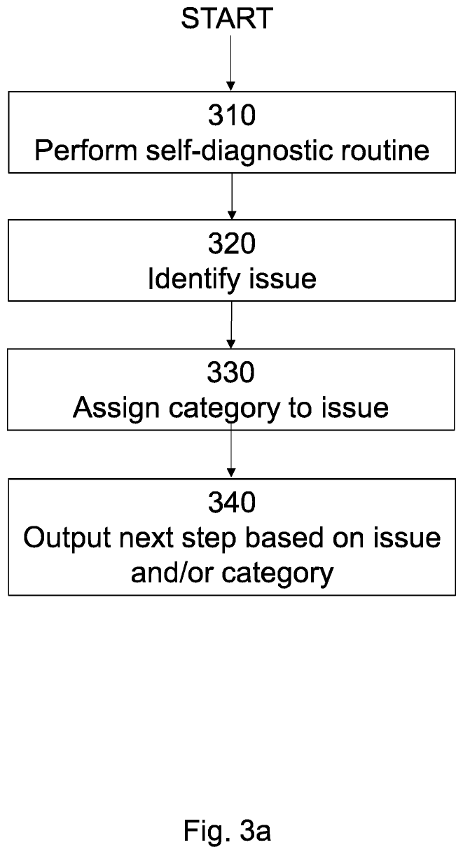

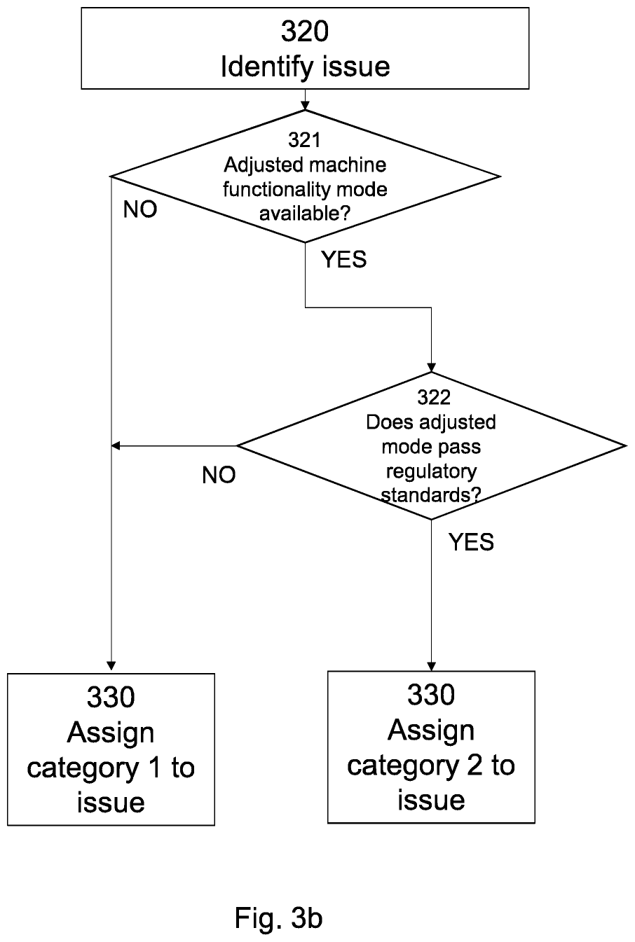

With reference to the flowchart depicted in FIGS. 3a and 3b, at step 310 data relating to an operational state of the radiotherapy device or a component thereof is obtained; in the embodiment shown the data is obtained when a diagnostic routine or process is performed but the data may alternatively be obtained by a sensor or detector in continuous operation or in any other suitable manner. This routine or process may be performed by any of the device controller 240, the central processor 270, the remote controller 260, or a combination of these processors.

At step 320, it is determined whether the radiotherapy machine has one or more faults, or issues, or a change in state or condition which may affect its operational performance or its operational longevity, and/or the performance or longevity of a particular component of the radiotherapy device.

Despite precision engineering and rigorous testing, some component parts of a radiotherapy device may start to degrade over its lifetime. If at any point during treatment a radiotherapy device starts to function outside of its normal operating parameters, a safety override or “interrupt” occurs. Unplanned equipment downtime can disrupt planned treatment schedules, and may be expensive for the machine owner, be it due to loss of revenue, servicing and repair costs, or both.

Diagnosing the fault may therefore comprise determining whether a safety override or “interrupt” has occurred, and may additionally comprise determining which type of safety override. The safety interrupt may be associated with a particular component of the radiotherapy device.

For example, if the position of a multi-leaf collimator (MLC) leaf during treatment as determined by an optical sensing system does not match the expected position of the MLC leaf as determined from signals supplied to the actuation mechanism configured to move the MLC leaf, a safety interrupt occurs and treatment is halted. This type of fault is very rare but must be planned for.

Diagnosing a fault with the radiotherapy machine may comprise receiving signals, comparing those signals to one or more operational thresholds, and determining that the machine is operating outside its normal safety parameters. The signals may be received from one or more different sensors in connection with the radiotherapy machine, and may measure, for example, the voltage, current, power, or any other feature associated with electrical power supplied to one of the components. These components may include the source of electrons such as an electron gun, a source of RF energy such as a magnetron, one or more vacuum pumps which are each configured to keep the vacuum system of the device at UHV conditions, one or more temperature sensors associated with the cooling system, one or more sensors associated with a dosimeter which measures the dose supplied by the beam of therapeutic radiation, one or more sensors associated with the position of beam limiting apparatus such as diaphragms and a multi-leaf collimator, sensors associated with the movement of the heavy metal target into and out from the path of the beam for the purpose of switching between a beam of therapeutic X-rays and a beam of therapeutic electrons, and/or one or more sensors associated with any of the components of the device mentioned above.

### Electron Gun Diagnostic Method

The self-diagnostic routine may comprise determining whether a component is nearing the end of its operational lifetime. By way of a specific example, it has been shown that signals from a current sensor associated with the electron gun can be monitored, and if it is determined that the current value meets at least one threshold criterion (for example has fallen below a threshold value), and if it is additionally determined that the current value has changed by at least a threshold amount in a particular time period, then it can be determined that the electron gun is nearing the end of its operational lifetime and that repair or replacement of the electron gun cathode should be scheduled.

### Flight Tube and Vacuum System Diagnostic Methods

By way of a further specific example, the self-diagnostic routine may comprise a method of determining the nature of a fault in a radiotherapy device comprising a vacuum tube or vacuum system comprising at least an electron gun, a waveguide configured to accelerate electrons emitted by the electron gun toward a target to produce said radiation, and a flight tube, the electron gun being located at a first end of the vacuum tube and the flight tube being located at a second end of the vacuum tube. By processing signals from one or more sensors which are configured to provide signals indicative of pressure within the vacuum tube, and by setting up signal thresholds in the correct way, it is possible to determine not only that the fault is associated with the vacuum system but also identify the specific region of the vacuum system, such as whether there is a leak in the vicinity of the electron gun, or the flight tube, or whether one or more of the vacuum pumps may have developed a fault. For example, by processing signals from a first and second sensor, the first sensor configured to provide signals indicative of pressure at a first region inside the vacuum tube and the second sensor being configured to provide signals indicative of pressure at a second region inside the vacuum tube, the first region being closer to the first end of the vacuum tube than the second region is, it is possible to determine whether there is a fault associated with the flight tube of the vacuum system. The diagnostic routine may comprise processing a first value derived from signals from the first sensor and a second value derived from signals from the second sensor, the first value being indicative of pressure at the first region inside the vacuum tube, and the second value being indicative of pressure at the second region inside the vacuum tube. Processing the first and second values may comprise comparing the first value with a first threshold and comparing the second value with a second threshold, and based on the processing of the signals, determining that there is a fault with the vacuum system of the device. In particular, comparing the second value with the second threshold may comprise determining that the second value is greater than the second threshold and comparing the first value with the first threshold may comprise determining that the first value is less than the first threshold. The process then moves to step 330 as discussed further below.

### Thyratron

In another example, a thyratron causes diode overload at high treatment energies.

### Magnetron Energy

In another example, a magnetron running in a tuning cavity is not able to generate the specific radio-frequency required to achieve a beam at a certain energy.

### Ion Chamber Measurements

In another example, instability in the ion chamber measurements leads to dosimetry error at some energies.

### Multi-Leaf Collimator

In another example, an issue may occur within a beam limiting device such as the multi-leaf collimator. For example, a bank of leaves or certain individual leaves may stop functioning and lock into place.

### Gantry Rotation Speed

In another example, an issue may occur which causes a reduction in gantry rotation speed, such as an issue with the gantry motor.

### Multi-Leaf Collimator Motor Speed

In another example, an issue may occur which causes a reduction the speed of individual leaf movement in the multi-leaf collimator (MLC), such as an issue with MLC motors. This may be observed by undue “pausing” of the gantry as the gantry waits for the MLC leaves to reach the correct positions.

As the skilled person would appreciate, any appropriate known systems may be used to detect certain faults in the thyratron, magnetron, ion chamber measurements, multi-leaf collimator or gantry, such as the faults described above. Such systems may comprise detectors, sensors performance monitors or other mechanisms or processes for identifying changes in operation as will be well known to the skilled person.

### Seasonal Shift

In another example, an issue may be detected according to steps 310 and 320 which correlates with a long-term seasonal shift. Radiotherapy machines go through a seasonal shift due to changes in humidity or temperature over a yearly time period. At step 320, an issue may be flagged according to any of the described methods above in correlation with changes in seasonal environmental conditions such as temperature and humidity. The correlation may be identified by monitoring of the date of fault recordal to identify variations over time, and with temperature and humidity sensors.

### Imaging Systems

In another example, imaging panels may have an issue, perform sub-optimally and cause deteriorated image quality. Equally, another part of the imaging system, such as a kV radiation source, may have an issue and cause deteriorated image quality. Image quality may be measured by regular (e.g. day to day or week to week) image analysis to track whether image quality is changing over time. For example this can be by comparing a measure of image quality, for example using a standard or control image, over a period to detect changes in quality over time.

At step 330, the radiotherapy machine may optionally look up a category associated with the issue. This may comprise consulting a category look-up table based on the issue or fault identified at step 320. A suitable look-up table is as follows:

| Type of fault/identified | Category of | |
|---|---|---|
| issue | fault | Associated next step(s) |
| The MLC leaves are not | Category 1 | Communicate nature |
| moving into the positions | | of fault to central |
| instructed by the device | | controller |
| processor. | | Output nature of fault |
| The linear accelerator is | | to device controller |
| overheating when the beam | | Halt or disallow all |
| is ‘on’. | | device operation. |
| | | Only allow over-ride/ |
| | | a return to service |
| | | when authorised by a |
| | | certified field service |
| | | engineer |
| The magnetron tuning cavity | Category 2 | Communicate nature |
| unable to produce an output | | of fault to central |
| at a specific energy (other | | controller |
| energies working without | | Output nature of fault |
| problems) | | to device controller |
| Thyratron causing diode | | Enter device into - |
| overload at certain energies | | fault specific |
| | | adjusted functionality |
| | | mode (discussed |
| | | further below) |
| The electron gun is nearing | Category 3 | Communicate nature |
| the end of its operational | | of fault to central |
| lifetime | | controller |
| | | Output nature of fault |
| | | to device controller |
| | | Provide walk-through |
| | | at the device |
| | | controller of how to |
| | | repair or replace the |
| | | component |
| Gantry stops intermittently | Category 4 | Communicate nature |
| Leak on the gas circuit | | of fault to central |
| Leak on internal water | | controller |
| system | | Output nature of fault |
| | | to device controller |

At step 340, the radiotherapy machine looks up the next step(s) associated with the category associated with the issue. This may comprise consulting the same category look-up table as at step 320. A suitable look-up table is therefore as provided above.

Additionally or alternatively, assigning a category associated with the issue at step 330 may comprise the process in FIG. 3b which depicts a flowchart which shows a method according to the present disclosure. The method distinguishes between a category 1 issue and a category 2 issue, between steps 320 and 330.

At step 320, it is determined whether the radiotherapy machine has one or more faults, or issues, which may affect its operational performance or its operational longevity, and/or the performance or longevity of a particular component of the radiotherapy device. At step 321, the issue is evaluated as to whether an “adjusted mode” of functionality is available. An adjusted mode of functionality must be able to complete treatment delivery in some capacity, despite the presence of a fault. In an adjusted mode of functionality, it is expected that the machine would be working at a reduced or different operability. When in an adjusted mode, the machine may work at an essential level of performance rather than at a nominal level of performance. If there is no adjusted mode available, it may require the halt of all treatment until the device has been fixed by an authorised engineer and is therefore assigned as a category 1 issue at step 330. If an adjusted mode of functionality is available, category 2 may be assigned and the method continues to step 322.

At step 322, after an adjusted mode of functionality is determined to be available, the mode is checked against regulatory standards. When a component or system of the machine is in an adjusted mode, regulatory standards relating to that component or system are checked. If regulatory standards are not met, it will require the halt of all treatment until the device has been fixed by an authorised engineer and is therefore assigned as a category 1 issue at step 330. If regulatory standards are met, the machine may be able to operate in its adjusted mode, in a reduced or different functionality state, and a category 2 is assigned to the issue at step 330. An adjusted mode allows the hospital to continue treating patients. Thus, downtime is avoided while a service of the device is planned permitting existing treatment plans to be continued safely.

Optionally, a message to the user may be delivered after step 322. The message may inform the user of the fault and of the proposed adjusted mode which the machine will operate in until the service of the device is complete. The message to the user may also ask the user whether they would like to proceed with the adjusted mode or not. If the user chooses not to proceed, the issue is assigned as category 1 at step 330. If the user chooses to proceed, the issue is assigned as category 2 at step 330.

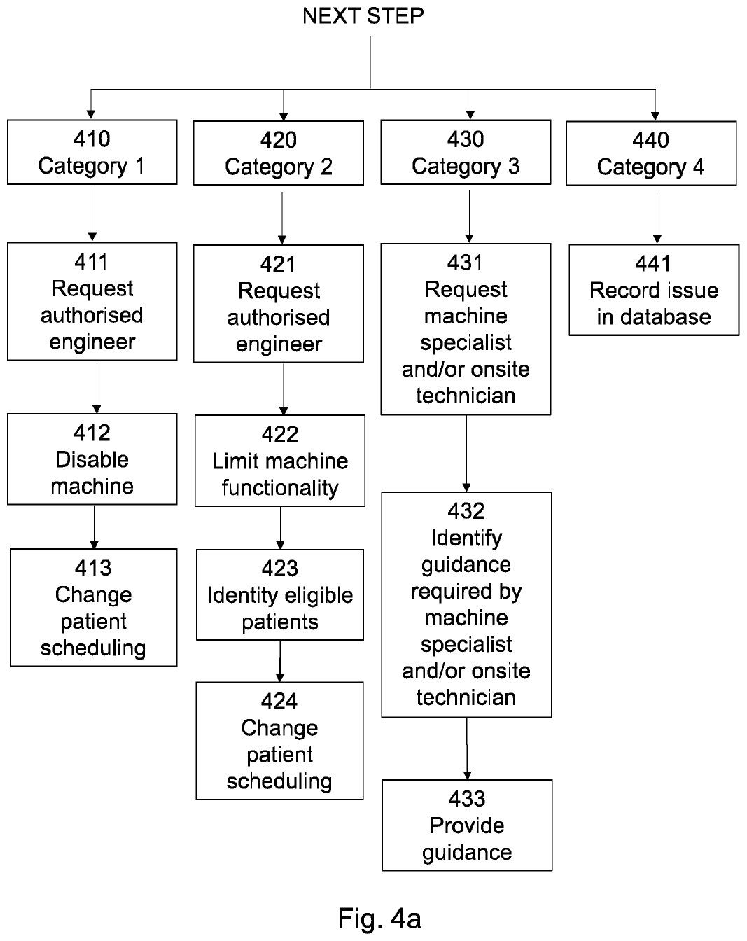

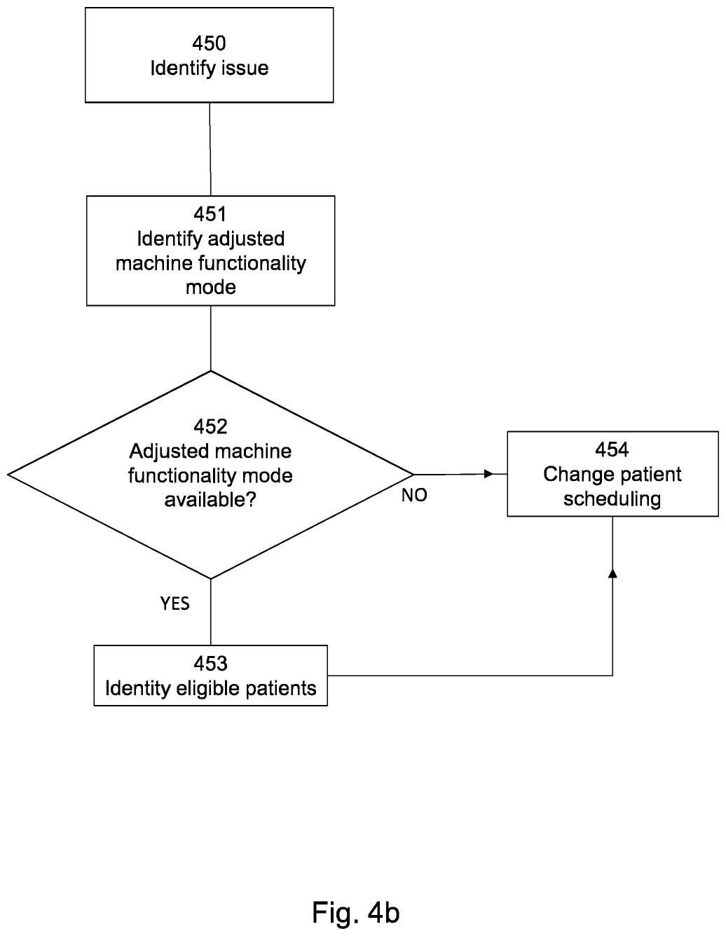

FIG. 4a depicts a flowchart which shows further examples of possible next steps according to the category assigned to an issue or fault at step 330.

All faults—regardless of category—are reported to the central server to be stored on a manufacturer's or service provider's database. In an implementation, all faults and/or issues identified are outputted to the device controller 240 in order to alert on-site staff to the results of the diagnostic process(es).

### Category 1: Taking the Device Out of Service—Machine Downtime

At step 410, the issue is identified as being a category 1 issue. This is the most severe category of issue. In this example, the corresponding next steps (based on the issue being a category 1 issue) are to request the support of an authorised engineer, as shown in step 411; and to disable the machine, as shown in step 412.

Issues may be identified as category 1 if they are severe or serious. For example, if the fault is of a severe or serious nature, it may be necessary to halt all treatment until the device has been serviced by a field service engineer (FSE) and preferably an FSE that has been certified by the device manufacturer as competent to repair this type of fault. These types of fault are very rare but must be planned for. In these instances, device downtime is inevitable, however by diagnosing the issue and informing the central processor immediately, servicing can be scheduled with the minimum of delay.

Category 1 issues include faults in the dosimetry channel which mean that the dosimetry is not at the expected values. Examples of such faults include: the magnetron being powerless; ion chamber leakages; and obstructions in the bending magnet coils.

At step 413, changes in patient scheduling may take place due to a category 1 issue. Patient treatments scheduled with a radiotherapy device exhibiting a category 1 issue may be cancelled or postponed until the authorised engineer has fixed the issue. For facilities with more than one radiotherapy device, it is likely that only one device would experience a category 1 at one instance in time. Patient treatments may be rescheduled using a prioritization system, for example prioritizing the most time sensitive treatments to be scheduled first. Changing patient scheduling may involve moving patient treatments forward or backwards in time. Changing patient scheduling may also involve changing the allocated radiotherapy machine for a particular patient treatment.

### Category 2: Adjusted Functionality Mode

At step 420, the issue is identified as being a category 2 issue. This is the second most severe category of issue. In this example, the corresponding next steps (based on the issue being a category 2 issue) are to request the support of an authorised engineer, as shown in step 421; and, for example in parallel or while awaiting the engineer, to limit or adjust the machine functionality, as shown in step 422. Limiting or changing the machine functionality may be referred to as placing the machine in an adjusted functionality mode.

In more detail, depending on the type or nature of the identified fault or issue, an appropriate next step may be allowing only reduced operability of the radiotherapy device. This allows the machine to operate in a reduced functionality state, while allowing the hospital to continue treating patients. Thus, downtime is avoided while a service of the device is planned.

As for the category 1 issue described above, the authorised engineer may be an FSE and preferably an FSE that has been certified by the device manufacturer as competent to repair the type of fault or issue identified.

This could incorporate a “health” score based on an assessment of different components and the state of disrepair.

The health score could be calculated for the number of alarms and the severity category of those alarms. The health score can be calculated based on the health status of each part of the LINAC. Each critical part i, has a prediction of RUL (residual useful life), a total expected useful life (EUL) and its corresponding weight (w) in the total machine health score based on the risk contribution of the part. The equation for computing LINAC Health Score (LHS) is:

$$L H S = ( λ \cdot {\prod }_{i} \frac{R U {L}^{i}}{E U {L}^{i}} \cdot {w}^{i} + ( 1 - λ ) \cdot \frac{total expected alarms scores}{weighted sum of alarms scores} ) \times 100$$

A LINAC with a low health score could have and of: a high risk of part malfunction; low performance; and a high risk of treatment interruptions which will delay the patient workflow.

A health score below a predetermined threshold value may be categorised as a low health score.

If a device is determined to have a low health score, proactive intervention is taken depending on the severity of the alarm and the number of interruptions. The individual health status of the critical components is checked in order to determine the root cause of the low health score.

If needed, field engineer visits are ordered/scheduled, or the components are checked during the next preventive maintenance.

Checking the health status of the critical components may include checking whether any of the components is near the end of its life. If it is determined that there is no part near end of life and the general alarm term results in the low score, issues may be resolved in the next preventive maintenance health check and routine maintenance. If there are critical parts near the end of life, a number of steps can be taken, including: informing the customer, ordering the parts and sending a field service engineer over to replace the parts.

Some faults could be addressed by the device entering an adjusted functionality mode.

Faults which can be addressed by an ‘adjusted functionality mode’ include faults in high temperature components of the LINAC, such as the magnetron or the thyratron,

When in the adjusted functionality mode, some elements of device functionality are retained, whereas some elements are disabled. The specific adjusted functionality mode may be issue or component dependent. For example, if it is determined that a specific energy negatively impacts the fault in the component in question (for example the magnetron or the thyratron), this energy is disabled. Further use of this energy may cause further damage to the component. An energy which does not negatively impact the fault in the component is enabled.

The device can continue to operate in ‘adjusted functionality mode’ operating an essential level of functionality until a time at which the device can be returned back to full functionality. An essential level of functionality requires that all regulatory standards are met for a particular treatment. In other words, a core level of functionality is maintained without any compromise to the quality of patient treatment. In an adjusted functionality mode, certain treatments may not be able to be conducted by the device whilst other treatments can continue without compromise to treatment quality. To bring the device back to full functionality the component is repaired, adjusted or replaced when the machine is not in use. QA controls will then be performed by authorised personnel to ensure the device is functioning within the required limits.

Further specific adjusted functionality mode examples will now be described:

### Thyratron

In one example, a thyratron causes diode overload at high treatment energies. In this case, the high treatment energies are disabled, and the LINAC continues to operate using only low energies until the component is replaced. Disabled high beam energy does not affect the use of the other low beam energies available. At step 423, identifying eligible patients may include processing patient treatment plans to determine which patient treatment plans do not require the disabled beam energies.

At step 424, changes in patient scheduling may take place. For facilities with more than one radiotherapy device, it is likely that only one device would experience an issue on thyratron causing diode overload at high treatment energies at one instance in time. Patient treatment plans which need the disabled beam energies but were scheduled on a first (disabled) device may be swapped for their treatment with a patient which does not require the disabled beam energy on a second (fully functioning) device. In some cases, patient treatments which require the disabled beam energies may be cancelled or postponed until the authorised engineer has fixed the issue. Patient treatments which do not require the disabled beam energies may continue to take place before the authorised engineer has fixed the issue. Patient treatments which do not require the disabled beam energy may move forward in time when other patient treatments are cancelled or postponed. At step 424, changes in patient scheduling are considered until the point at which the issue has been resolved.

### Magnetron Energy

In another example, a magnetron running in a tuning cavity is not able to generate the specific radio-frequency required to achieve a beam at a certain energy. This beam energy is disabled and the LINAC continues generating beams at energies which are not disabled. One specific disabled beam energy does not affect the use of the other beam energies available. At step 423, identifying eligible patients may include processing patient treatment plans to determine which patient treatment plans do not require the disabled beam energy.

At step 424, changes in patient scheduling may take place. For facilities with more than one radiotherapy device, it is likely that only one device would experience an issue on a specific beam energy at one instance in time. The same steps may be applied as described above for the thyratron example.

### Ion Chamber Measurements

In another example, instability in the ion chamber measurements leads to dosimetry error at some energies. The energies at risk of dosimetry error are disabled. Similarly to the issue where the magnetron is not able to generate a specific beam energy, energies disabled due risk of dosimetry error due to ion chamber measurements does not affect the use of the other beam energies available. At step 423, identifying eligible patients may include processing patient treatment plans to determine which patient treatment plans do not require the disabled beam energy.

At step 424, changes in patient scheduling may take place. For facilities with more than one radiotherapy device, it is likely that only one device would experience an issue on a specific beam energy at one instance in time. The same steps may be applied as described above for the thyratron example.

### Imaging Systems

In another example, imaging panels may have an issue, perform sub-optimally or differently and cause deteriorated image quality. Equally, another part of the imaging system, such as a kV radiation source, may have an issue and cause deteriorated image quality. It is possible for clinicians to continue to work with degraded image quality within a range of quality levels, hence at step 423, identifying eligible patients may include processing patient treatment plans to determine which patients need imaging. Some patients may not require imaging in the delivery of their treatment, therefore a degraded imaging system may not affect delivery. In some cases, the issue associated with the imaging system may cause a deterioration in image quality where the image is sufficient for registration and repositioning but not sufficient to provide images for treatment planning. Patients treatment which need only image registration may still be treated in circumstances where the imaging panel or system is degraded but still functioning in registration.

At step 424, changes in patient scheduling may involve prioritising patients who do not require use of the imaging system. If the imaging system is able to complete registration, then patients which require only registration and repositioning for their treatments may also be prioritised. In some cases, if the registration system is at fault but the image quality is sufficient, then the images may be sent via a remote system (e.g. via the remote access system described in FIG. 2) to be registered at a remote location. Patient treatment plans which need use of an imaging system but were scheduled on a first (image quality degraded) device may be swapped for their treatment with a patient which does not require the disabled imaging system on a second (fully functioning) device. In some cases, patient treatments which require full use of the imaging system (e.g. for further treatment planning) may be cancelled or postponed until the authorised engineer has fixed the issue. Patient treatments which do not require use of the imaging system may move forward in time when other patient treatments are cancelled or postponed. At step 424, changes in patient scheduling are considered until the point at which the issue has been resolved.

### Multi-Leaf Collimator

In another example, an issue may occur within a beam limiting device such as the multi-leaf collimator. For example, a bank of leaves or certain individual leaves may stop functioning and lock into place. However, certain patient treatment plans may still be able to be delivered without comprising the delivery of the treatment. For some treatments, not all leaves of multi-leaf collimator may be required to function. A treatment plan may correspond to delivery of a small treatment beam to the patient and not use one bank of leaves, for example. In some cases, treatment plans may be able to be translated to the functioning leaves of the MLC and be able to be delivered to the patient.

At step 423, identifying eligible patients may include processing patient treatment plans to determine which patient treatment plans do not require the leaves or banks of the multi-leaf collimator which are at fault. Identifying eligible patients may also include processing patient treatment plans to determine if any treatment plans may be translated into functioning leaves or banks of the MLC.

At step 424, changes in patient scheduling may take place. For facilities with more than one radiotherapy device, it is likely that only one device would experience an issue with the MLC at one instance in time. Patient treatment plans which need the MLC leaves or banks at fault but were scheduled on a first (disabled) device may be swapped for their treatment with a patient who does not require the leaves or banks at fault on a second (fully functioning) device. In some cases, patient treatments which require the leaves or banks at fault with the MLC may be cancelled or postponed until the authorised engineer has fixed the issue. Patient treatments which do not require the leaves or banks at fault with the MLC may continue to take place before the authorised engineer has fixed the issue. Patient treatments which do not require the leaves or banks at fault with the MLC may move forward in time when other patient treatments are cancelled or postponed. At step 424, changes in patient scheduling are considered until the point at which the issue has been resolved.

### Gantry Rotation Speed

In another example, an issue may occur which causes a reduction in gantry rotation speed, such as an issue with the gantry motor. A reduction in gantry rotation speed will not degrade the quality of treatment to the patient but may take longer to complete the treatment. At step 423, identifying eligible patients may involve identifying that the majority of patients are eligible for their treatments to continue at reduced speed. At step 424, changes in patient scheduling may involve making adjustments due to the increased patient treatment time for each patient. Fewer patients may be able to be treated in a day, until the authorised engineer has fixed the issue.

### Multi-Leaf Collimator Motor Speed

In another example, an issue may occur which causes a reduction in the speed of individual leaf movement in the multi-leaf collimator (MLC), such as an issue with MLC motors. A reduction in MLC motor speed will not degrade the quality of treatment to the patient but may take longer to complete the treatment. To aid delivery in such a case, the gantry rotation may be deliberately slowed to match the slowed MLC leaf speed. At step 423, identifying eligible patients may involve identifying that the majority of patients are eligible for their treatments to continue at reduced speed. At step 424, changes in patient scheduling may involve making adjustments due to the increased patient treatment time for each patient. Fewer patients may be able to be treated in a day, until the authorised engineer has fixed the issue.

### Seasonal Shift

In another example, an issue may be detected according to steps 310 and 320 which correlates with a long-term seasonal shift. Radiotherapy machines go through a seasonal shift due to changes in humidity or temperature over a yearly time period. After a change is detected or predicted, the environment of the radiotherapy device may be automatically controlled via humidity or temperature control, for example, aircon systems. If parts of the radiotherapy machine overheat, these parts may be shut down or limited until the temperature it is within required levels. At step 423, identifying eligible patients may involve identifying that the majority of patients are eligible for their treatments to continue. At step 424, changes in patient scheduling may involve making little to no adjustments.

In general, at step 424, changing patient scheduling may involve moving patient treatments forward or backwards time. Changing patient scheduling may also involve changing the accolated radiotherapy machine for a particular patient treatment.

At step 424, any category 2 issue may optionally include sending a message to the user. The message may inform the user of the fault, of the proposed adjusted mode and the proposed changes to patient scheduling. The message to the user may also ask the user whether they would like to proceed with the changes to patient scheduling or not. In some cases, particular rooms may contain equipment external to the radiotherapy device and necessary for certain treatment such as accessories or masks.

FIG. 4b depicts a method according to the present disclosure for a category 2 issue. The method depicted in FIG. 4b is in accordance with the methods described in FIGS. 3a, 3b and 4a. At step 450, data relating to an operational state of the radiotherapy device or a component thereof is obtained and an issue is identified with the radiotherapy device based on the obtained data. At step 451, an adjusted functionality mode of the machine is identified based on the issue identified at step 450. At step 452, it is determined whether an adjusted machine functionality mode is available. If there is an adjusted mode available, step 453 is executed and eligible patients are identified. Eligible patients may include patients with treatment plans which may still be carried out with the machine in the adjusted functionality mode. At step 454, changes in patient scheduling are made. If there is an adjusted mode available, step 454 may include swapping patients treatments between fully-functioning and adjusted machines. If there is no an adjusted mode available, step 454 is executed and step 454 may include postponing patient treatments.

### Category 4—Log the Issue for Check-Up During Next Scheduled Maintenance/Servicing Point

At step 440, the issue is identified as being a category 4 issue. This is the least severe category of issue. In this example, the corresponding next step (based on the issue being a category 4 issue) is to record the issue in a database, as shown in step 441. In other words, no further action is taken beyond the default next step carried out for all categories of issue: that being to report the issue or fault to the central server to be stored on a manufacturer's or service provider's database. The issue or fault may then form part of the maintenance plan at the next scheduled service of the machine by, for example, an FSE or onsite technician. In the meantime, the machine continues to function as normal i.e. with no reduction in operability.

One example of a category 4 issue is sporadic gantry stops. These stops do not halt the treatment but could generate treatments delays. Another category 4 issue is a demand for thumbwheels for use in a patient support system or a hand-held controller. Such demands could generate treatments delays (the frequency or number of alarms could be used to predict the demand for thumbscrews).

In these category 4 situations the issue is noted and logged for a check-up during the next scheduled maintenance or service.

### Category 3—Coaching Staff on how to Fix the Problem

At step 430, the issue is identified as being a category 3 issue. This is the second least severe category of issue. In this example, the corresponding next step (based on the issue being a category 3 issue) is to request a machine specialist and/or onsite technician, as shown in step 431; to identify guidance required by the machine specialist and/or onsite technician, as shown in step 432; and to provide the guidance, as shown in step 433.

The machine specialist and/or onsite technician may be, for example, a hospital employee specially trained in operating and resolving some issues with the machine. The machine specialist and/or onsite technician may though be less qualified and/or experienced than an FSE and so may only be authorised to fix certain issues with the machine.

In one example, the guidance referred to in steps 432 and 433 is guidance for implementing a next step for overcoming the identified issue with the machine. The guidance may be provided on screen at the device controller 240. Alternatively, or additionally, the guidance may be provided on a user device of the machine specialist and/or onsite technician, such as a mobile phone, laptop or augmented reality headset. The acquisition and provision of guidance is described in more detail below.

As set out below, guidance may also be provided if a category 1 or 2 issue is identified at step 330. In that case, guidance may be provided to the authorised engineer.

### Provision of Guidance: Sensor Informed Instructions

In one example, the guidance comprises a series of steps for overcoming the identified issue with the machine. A next step in the series is only communicated when the preceding step has been successfully implemented by, for example, an onsite technician. Successful implementation of a step is determined by one or more of the sensors 230 distributed throughout the machine. During provision of the guidance and/or implementation of the steps for overcoming the issue, the device controller 240 may limit the machine functionality or fully disable the machine. When the machine is fully disabled, the plurality of sensors 230 distributed around the machine may still operate and communicate with the device controller 240 to provide feedback on the machine's operability.

An example of a fault that could be solved via a walk-through provided to hospital staff at the device is filling up the internal water press and gas press following a leak on the water circuit or the gas circuit. Such actions can be performed by a member of the hospital staff upon instruction.

By way of a specific example, a category 3 issue is identified at the machine: the issue being that a fuse of the machine has blown. A first step in resolving the issue is provided on a screen at the device controller 240. The first step includes instructions for opening a given hatch of the machine. An onsite technician considers and implements this first step, i.e. the onsite technician opens the machine hatch. When the first step has been implemented, a sensor 230 associated with the hatch determines that the hatch has been opened and communicates this to the device controller 240. In this way, the sensor 230 associated with the hatch confirms that the first step has been successfully implemented. In response, the device controller 240 provides a second step in resolving the issue. The second step includes instructions for removing the blown fuse, the blown fuse being located behind the hatch. The onsite technician considers and implements this second step. As for the first step, when the second step has been implemented, a sensor 230 associated with the fuse determines that the fuse has been removed and communicates this to the device controller 240. In this way, the sensor confirms that the second step has been successfully implemented. In response, the device controller 240 provides a third step in resolving the issue. The third step includes instructions for installing a new fuse. An onsite technician considers and implements this third step. As for the first and second steps, when the third step has been implemented, a sensor 230 associated with the fuse location determines that a new fuse has been installed and communicates this to the device controller 240. In response, the device controller 240 provides a fourth—and final—step in resolving the issue. The fourth step includes instructions for closing the hatch covering the fuse. The onsite technician considers and implements this fourth step. As for the preceding steps, when the fourth step has been implemented, the sensor 230 associated with the hatch determines that the hatch has been closed and communicates this to the device controller 240. In this way, the sensor confirms that the fourth—and final—step has been successfully implemented and—upon successful completion of a self-diagnostic route—the device controller 240 concludes that the issue has been overcome.

The example provided in the preceding paragraph is intended as a specific example of the fundamental principles of the sensor informed instruction approach and modifications may be made as will occur to the skilled person. In particular

The specific issue may be of any kind as will occur to the skilled person. There may be any number of steps in the guidance for overcoming the issue. The issue resolution may be informed by any number of the plurality of sensors 230 distributed around the machine. The steps may be implemented by a ‘lay person’, and—for example—not an onsite technical or machine specialist.

The guidance may be stored in the device controller memory 245. Alternatively or additionally, the guidance may be received from a remote location. The supply of guidance from a remote location is discussed in detail in the sections below.

The guidance may be provided to the person implementing the next steps in various ways. For example, the guidance may be provided to the person implementing the next steps on a single user device or a combination of user devices, such as a mobile phone, laptop, virtual reality headset and/or augmented reality headset. Additionally or alternatively, the guidance may also be provided on a screen at the device controller 240, as described above. The guidance may be communicated in any format, for example: the guidance may be in an audio and/or visual format.

References in this disclosure to ‘next step’ may be understood to be references to one, or a series of next steps for overcoming an issue which are deployable in the above described sensor informed approach.

### Provision of Guidance: Centralising the Competency

### 1. General Overview

Systems and methods are described here for providing a field-service engineer, machine specialist and/or onsite technician, who is on site at a user location, with instructions for installing, servicing, repairing, and/or maintaining a radiotherapy device at the user location. The instructions are issued from a site remote from the user location. Competency relating to the installation and/or maintenance of the radiotherapy device is centralised at the remote site and that competency communicated out to each of the user locations where it is required. As a result, and as is explained in more detail below, problems in the prior art are addressed. The radiotherapy device or devices may comprise any of a linear accelerator (linac), an MRI machine, and an MR linac.

In an example, the competency centralised at the remote site comprises at least one, and perhaps more, engineers experienced in the art of installing and/or servicing or repairing radiotherapy devices. When a user, such as a member of staff at a hospital where the device is installed or a less experienced field-service engineer on site at the user location identifies an issue with the installation or maintenance of a radiotherapy device at the user location, he or she sends data indicative of the issue to the remote site. The experienced engineer(s) at the remote site receives the data; processes the data to understand the issue; and—in response—generates instructions for overcoming the issue. These instructions, which may be referred to interchangeably as guidance, are sent to the field-service engineer. Upon receipt, the field-service engineer implements the next step or steps outlined in the instructions and so may overcome the installation or maintenance issue.

In this way, problems in the prior art are addressed. This includes:
The amount of overall travelling is reduced as only one field-service engineer needs to be deployed to each user location (regardless of experience level) or, alternatively, a member of staff at the device location can repair the issue based on the engineer's instructions.
Training is improved as the most experienced engineers may remain at the remote site and provide guidance to less experienced engineers at the user locations.
Information exchange is vastly improved as all installation and maintenance issues may be reported to the remote site, which can help inform research and development for the radiotherapy devices and training for the field-service engineers, as well as ensure that service engineer repairs are implemented in a manner which is consistent across sites.
The quality of maintenance at each site can be improved, as a highly experienced engineer is able to oversee each installation, repair or part replacement. Thus, better service is provided and more efficient use is made of engineer time.

### 2. System Overview

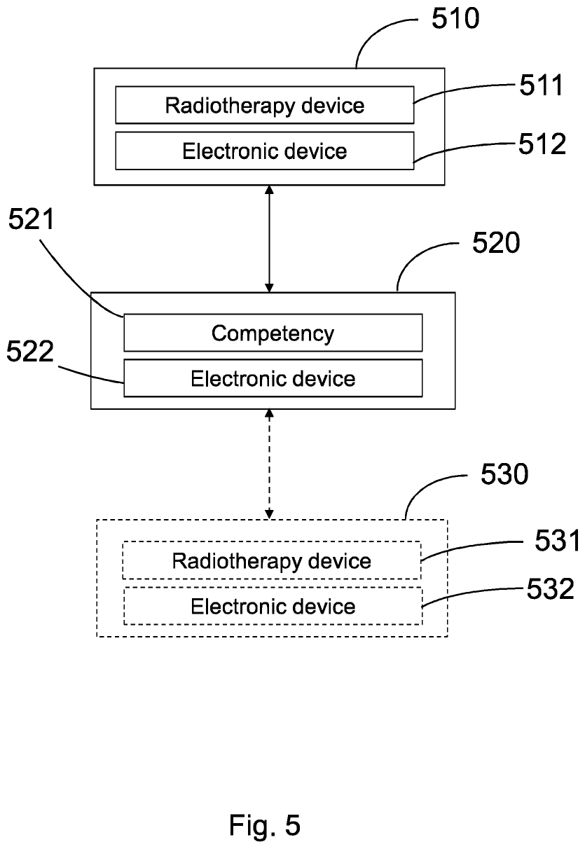

FIG. 5 is a schematic diagram of a system according to the present disclosure. The system comprises a first radiotherapy device 511 located at a first site 510. The first site 510 may be a hospital or a radiotherapy clinic, for example. The system further comprises a second site 520 remote from the first site 510. Competency 521 relating to the installation and/or manufacture of the first radiotherapy device 511 is centralised at the second site 520.

The system further comprises a first electronic device 512 located at the first site 510 and a second electronic device 522 located at the second site 520. The first and second electronic devices 512, 522 are communicatively coupled with each other such that the first electronic device 512 may transmit—that is, send—data to, and receive data from, the second electronic device 522, and vice versa. The first electronic device 512 comprises a first device controller. The second electronic device 522 comprises a second device controller. Both the first electronic device 512 and the second electronic device 522 may comprise a transmitter and a receiver.

The system may further comprise one or more further sites 530. Each of the one or more further sites 530 may have a further radiotherapy device 531 and a further electronic device 532 located at the respective site. Each further electronic device 532 may be communicatively coupled with the second electronic device 522 such that each further electronic device 532 may transmit—that is, send—and receive data to and from the second electronic device 522, and vice versa.

The first 512 and second 522 electronics devices may each be connected with a power source or provided with their own power supply, such as an internal battery.

Each of the radiotherapy devices 511, 531 shown in the figure comprises a linear acceleration (linac) device, and may further comprise an MR imager such that the radiotherapy device can be described as an MR linac.

The first and second electronic devices 512, 522 may each be described as processors, computers, or computing devices. The first and second electronic devices 512, 522 may be connected (e.g., networked) to each other and/or to other machines in a Local Area Network (LAN), an intranet, an extranet, or the internet. The first and second electronic devices may each operate in the capacity of a server or a client machine in a client-server network environment, or as a peer machine in a peer-to-peer (or distributed) network environment. The first and second electronic devices may each be a personal computer (PC), such as a laptop computer, a tablet computer, optionally provided with software such as Service360, a set-top box (STB), a Personal Digital Assistant (PDA), a cellular telephone, a smartphone, an augmented reality or mixed reality wearable, such as a Microsoft HoloLens, a web appliance, a server, a network router, switch or bridge, or any machine capable of executing a set of instructions (sequential or otherwise) that specify actions to be taken by that machine. Further, the term “electronic device” shall also be taken to include any collection of machines (e.g., computers) that individually or jointly execute a set (or multiple sets) of instructions to perform any one or more of the implementations and methodologies discussed herein.

A number of implementations and features of the system shown in FIG. 5 will now be described. In particular, the described implementations disclose different ways in which an engineer at the first site 510 can communicate an issue with the installation or maintenance of the first radiotherapy device 511 to the remote site 520.

### 3. First Implementation—Audio Guidance

In a first implementation, a first microphone device is provided at the first site 510 and a second microphone device is provided at the second site 520. In the first implementation, the first and second microphone devices are each comprised in the first and second electronic devices 512, 522, respectively. The first microphone device is communicatively coupled to the first device controller. Likewise, the second microphone device is communicatively coupled to the second device controller.

The first and second electronic devices 512, 522 further comprise first and second speakers, respectively. The first and second speakers are each communicatively coupled to the first and second device controllers, respectively. The first and second device controllers are each configured to receive and process the audio data acquired by the first and second microphone devices, respectively. Additionally, the first and second device controllers are each configured to output audio data to the first and second speakers, respectively.

In an example of the first implementation, one or both of the first and second speakers may instead be an audio port arranged to connect to an audio device. The audio device may comprise a speaker, speakers or headphones.

In operation, a first engineer at the first site 510 identifies an issue with the maintenance or installation of the first radiotherapy device 511. The first engineer inputs to the first microphone an audio description of the issue. The first microphone converts the sound waves created by the audio description into an electric signal. The electric signal is audio data identifying the issue with the first radiotherapy device 511. The audio data identifying the issue is received by the first data controller and is processed by the first data controller. On receipt of instructions to do so (for example, on receipt of a particular user input to the first electronic device 512), the audio data identifying the issue is sent to the second electronic device 522 at the second site 520. The audio data identifying the issue is received and processed by the second data controller comprised in the second electronic device 522. On receipt of an instruction to do so, the audio data identifying the issue is sent to the second speaker. The second speak outputs the audio data identifying the issue as an audio output. In this way, a second engineer at the second site 520 may listen to the first engineer's audio description of the issue.

In response to the first engineer's audio description of the issue, the second engineer inputs to the second microphone an audio description of a next step for installation or maintenance of the first radiotherapy device 511. The second microphone converts the sound waves created by the audio description into an electric signal. The electric signal is audio data identifying the next step for the installation or maintenance of the first radiotherapy device 511. Thus, audio data is generated identifying the next step for installing or maintaining the first radiotherapy device 511 based on the audio data received from the first electronic device 512. The audio data identifying the next step is received by the second data controller and is processed by the second data controller. On receipt of an instructions to do so (for example, on receipt of a particular user input to the second electronic device 522), the audio data identifying the next step is sent to the first electronic device 512 at the first site 510. The audio data identifying the next step is received and processed by the first data controller. On receipt of an instruction to do so (for example, on receipt of a particular user input to the first electronic device 512), the audio data identifying the next step is sent to the first speaker. The first speaker outputs the audio data identifying the next step as an audio output. In this way, the first engineer at the second site 520 may listen to the second engineer's audio description of the next step. Finally, the first engineer implements the next step for the installation or maintenance of the first radiotherapy device 511. That is, the first engineer installs and/or maintains the radiotherapy device 511 based on the audio data identifying the next step received from the first engineer.

In an example according to the first implementation, the first and second electronic devices 512, 522 comprise voice translation software. When executed, the voice translation software is arranged to convert first audio data of a first language into second audio data of a second language. Accordingly, in this example, it is not essential that the first and second engineers speak and listen in the same language; instead, the first engineer may speak and listen in French, for example, while the second engineer may speak and listen in English, for example. Advantageously language barriers may thus be overcome.

As will be apparent to the skilled person, the first microphone, second microphone, first speaker and second speaker need not be comprised in the respective ones of the first and second electronic devices 512, 522. Instead, any of these devices may be comprised in a further electronic devices and may be wired or wirelessly communicatively coupled to the respective one of the first and/or second data controller.

### 4. Second Implementation—Video Guidance

In a second implementation, which may complement the first implementation, a first camera device is provided at the first site 510 and a second camera is provided at the second site 520. In the second implementation, the first and second camera devices are each comprised in the first and second electronic devices 512, 522, respectively. The first camera device is communicatively coupled to the first device controller. Likewise, the second camera device is communicatively coupled to the second device controller.

The first and second electronic devices 512, 522 each further comprise first and second displays, respectively. The first and second displays are each communicatively coupled to the first and second device controllers, respectively. The first and second device controllers are each configured to receive and process the image data acquired by the first and second cameras, respectively. Additionally, the first and second device controllers are each configured to output image data to the first and second displays, respectively.

In operation, a first engineer at the first site 510 identifies an issue with the maintenance or installation of the first radiotherapy device 511. The first engineer captures, using the first camera, an image or images which illustrate the issue with the maintenance or installation of the first radiotherapy device 511. For example, the image or images may illustrate a present condition of a particular component of the first radiotherapy device 511. The first camera converts the image or images into image data identifying the issue with the first radiotherapy device 511. The image data identifying the issue is received by the first data controller and is processed by the first data controller. On receipt of an instruction to do so, the image data identifying the issue is sent to the second electronic device 522. The image data identifying the issue is received and processed by the second data controller. On receipt of an instruction to do so, the image data identifying the issue is sent to the second display. The second display displays the image data identifying the issue. In this way, the second engineer at the second site 520 may view the image(s) showing the issue with the first radiotherapy device 511.

In response to the first engineer's image(s), the second engineer captures, using the second camera, an image or images which illustrate a next step for installation and/or maintenance of the first radiotherapy device 511. The second camera converts the image or images into image data identifying the next step. Thus, image data is generated identifying the next step for installing and/or maintaining the first radiotherapy device 511 based on the image data received from the first electronic device 512 identifying the issue. The image data identifying the next step is received by the second data controller and is processed by the second data controller. On receipt of an instruction to do so, the image data identifying the next step is sent to the first electronic device 512. The image data identifying the next step is received and processed by the first data controller. On receipt of an instruction to do so, the image data identifying the next step is sent to the first display. The first display displays the image data identifying the next step. In this way, the first engineer at the second site 520 may view the image(s) showing the next step for the installation or maintenance of the first radiotherapy device 511. Finally, the first engineer implements the next step for the installation or maintenance of the first radiotherapy device 511. That is, the first engineer installs or maintains the radiotherapy device 511 based on the image data identifying the next step.

In an example of the second implementation, the images(s) captured by the first and/or second camera are a sequence of images in time and together form a video. In that case, the term ‘image data’ disclosed above might equally well be replaced with ‘video data’. Accordingly, in this example, the first and second engineers may exchange video(s) showing (a) the issue with the installation or maintenance of the first radiotherapy device 511 and/or (b) the next step for installing and/or maintaining the first radiotherapy device 511.

In a further example of the second implementation, the image or video data is exchanged between the first and second electronic devices 512, 522 in substantially real-time. Advantageously, this means that the time from the first engineer identifying the issue to the first engineer implementing the next step is reduced as the communication lag is minimised. Accordingly, the time to installation or—in the case of maintenance—the downtime of the first radiotherapy device 511 is minimised.

### 5. Third Implementation—Audio and Image/Video Guidance

In a third implementation, the first and second implementations are combined such that both image/video data and audio data is exchanged between the first and second electronic devices 512, 522.

In an example of the third implementation, the image data is video data and the audio data is associated with a same time base as the video data. In operation, the video data and the audio data are displayed and output (that is, played) simultaneously. In this way, the first and second engineers may record videos with audio commentary and exchange this with each other. The video data and the audio data may be exchanged between the first and second electronic devices 512, 522 in substantially real-time. Advantageously, for similar reasons as discussed above, this means that the time from the first engineer identifying the issue to the first engineer implementing the next step is reduced. Accordingly, installation time or downtime (in the case of maintenance) of the first radiotherapy device 511 is minimised.

Overall, technical advantages of the first, second and third implementations include the following:
There is a low burden on the communication link between the first and second electronic devices 512, 522. This is because the image, video and/or audio data packages are reasonably modest in size. Thus, transmission of the data between the first and second electronic devices 512, 522 is also fast and reliable.
No specialist technical equipment is required. Instead, low cost electronic devices, such as mobile phones or tablet computers, may be used as the first and second electronic devices 512, 522. Such low cost electronic devices are typically provided with in-built microphones, speakers and cameras.
The use of image/video data improves safety as the second engineer has more evidence to base his or her assessment of the next step for installation or maintenance of the first radiotherapy device on. Additionally, the provision of image/video data provides the second engineer with the opportunity to see further issues not yet identified by the first engineer, such as components in need of maintenance or replacement, or to raise safety concerns.

### 6. Fourth Implementation—Diagnosis Using Augmented Reality (AR) or Mixed Reality

In a fourth implementation, the system further comprises, at the first site 510, one or more scanners communicatively coupled to the first data controller. Each scanner is configured to collect data about the environment within its respective range. The first data controller is arranged to receive the environment data and process the data to generate a digital three-dimensional model of the environment. In the fourth implementation, the system further comprises, at the second site 520, augmented reality rendering apparatus. The augmented reality rendering apparatus is communicatively coupled to the second data controller.

In operation, a first engineer at the first site 510 identifies an issue with the maintenance or installation of the first radiotherapy device 511. The first engineer scans, using the one or more scanners, the environment surrounding and including the first radiotherapy device 511, or the particular portion of the radiotherapy device 511 to which the issue relates. For example, the first engineer may scan the environment surrounding and including a particular component in the first radiotherapy device 511 which the first engineer has identified as problematic. In this way, the one or more scanners collect data about the environment surrounding and including the first radiotherapy device 511. The resulting environment data is received by the first data controller and is processed by the first data controller. Specifically, the first data controller may process the environment data to generate a three-dimensional model of the environment surrounding and including the first radiotherapy device 511. On receipt of an instructions to do so, the three-dimensional model is sent to the second electronic device 522. The three-dimensional model is received and processed by the second data controller. On receipt of an instruction to do so, the three-dimensional model is sent to the augmented reality rendering apparatus. The augmented reality rendering apparatus displays the three-dimensional-model to the second engineer. In this way, the second engineer at the second site 520 may ‘see’ the first radiotherapy device 511 as the first engineer sees it, and—specifically—may ‘see’ the issue with the first radiotherapy device 511.

In response to observing the three-dimensional model, the second engineer generates and returns data to the first electronic device 512 identifying the next step for the installation and/or maintenance of the first radiotherapy device. This may be done in accordance with any of the described implementations.

In an example of the fourth implementation, the one or more scanners are three-dimensional scanners.

The one or more scanners may be hand-held scanners. Alternatively, the one or more scanners may be attached to apparatus arranged to be worn by the engineer, such as eyeglasses, or attached to clothing, such as a t-shirt.

The augmented reality rendering apparatus may comprise an optical projection system or a display. The augmented reality rendering apparatus may be, or be comprised in, a handheld device. The augmented reality rendering apparatus may be, or be comprised in, apparatus arranged to be worn on the human body or attached to clothing.

In a specific example of the fourth implementation, the augmented reality rendering apparatus comprises a head-mounted display (HMD). The HMD is a display device arranged, in use, to be worn on the forehead of the second engineer. In use, the HMD displays the three-dimensional model to the second engineer by placing images corresponding to the three-dimensional model over the user's field of view.

In another specific example of the fourth implementation, the diagnosis described in this implementation is carried out using mixed reality. In this example, the phrase “augmented reality” used to describe this implementation can be replaced with the phrase “mixed reality”. For example, in this example, the augmented reality rending apparatus is mixed reality apparatus, such as a Microsoft HoloLens.

In some examples, the augmented reality rendering apparatus is comprised in the second electronic device. Advantageously, this is a low-cost solution as no specialist hardware is required at the second site 520.

By using AR, the first engineer's physical environment is virtually recreated in the second engineer's environment. As a result, the second engineer is provided with an immersive experience of what the first engineer is encountering at the first site 510. This means that the second engineer is provided with a much higher granularity of information to base his or her understanding of the issue with the first radiotherapy device 511 on. This has numerous technical advantages, including:
The time to install the first radiotherapy device 511 or, in the case of maintenance, the downtime of the first radiotherapy device 511 is significantly reduced. This is because the second engineer is able to confirm the issue with the first radiotherapy device 511 with a much higher degree of accuracy. Accordingly, the second engineer can issue instructions for a next step which has a high likelihood of addressing the issue. This improved likelihood of success reduces installation time and downtime.
The second engineer may base his or her assessment of the issue on the actual status of the first radiotherapy device 511, rather than on the first engineer's diagnosis. This improves both training and safety as the as the second engineer can correct oversights or incorrections in the first engineer's diagnosis.

### 7. Fifth Implementation—Virtually Mirroring the Issue

In a fifth implementation, on receiving data identifying the issue (the data being according to any of the disclosed implementations or combinations thereof), the second engineer virtually recreates or mirrors the issue. In this way, the second engineer can (a) verify the first engineer's diagnosis of the issue and (b) arrive at a next step which has a higher likelihood of success. Advantageously, because the issue is viewed in a greater level of context to the second engineer, the next step for overcoming the issue generated by the second engineer has a far higher likelihood of success and safety is improved as the second engineer is able to spot other issues with the machine.

In more detail, in an example of the fifth implementation, the second engineer virtually recreates the issue using computer-aided design (CAD) software. This is achieved by either (a) generating a two-dimensional or three-dimensional model of the first radiotherapy device with the issue in the CAD software—that is, generating the model from scratch; or (b) loading a previously generated two-dimensional or three-dimensional model of the first radiotherapy device 511 and manipulating the previously generated model to exhibit the issue. Once the model of the first radiotherapy device 511 with the issue has been generated, the second engineer determines—by interacting with the model in the CAD software, for example—the next step for overcoming the issue.

In an example, the second engineer may apply the next step to the model in the CAD software. That is, the second engineer may manipulate the model in the same way as the first engineer ought to manipulate the model to overcome the issue. In this case, the second engineer may record their interaction with the model and thus record a video of the next step. The video may then be sent to the first electronic device 512 for displaying on the first display, for example.

In another example of the fifth implementation, the second engineer virtually recreates the issue and optionally also generates implementation of the next step as augmented, mixed or virtual reality content. Each of these implementations will now be discussed in more detail:
Where the implementation of the next step is generated as virtual reality content, the virtual reality content is sent to the first electronic device 512. In this example, the system further comprises virtual reality rendering apparatus at the first site 510, such as a virtual reality headset. In this way, the first engineer is able to ‘explore’ and ‘interact’ with the virtual reality content representative of the first radiotherapy device and the next step for overcoming the issue. The virtual reality content representative of the next step for overcoming the issue may include data, such as installation or maintenance instructions. These instructions may be overlaid on the virtual reality content representative of the first radiotherapy device.
Where the implementation of the next step is generated as augmented or mixed reality content, the augmented or mixed reality content is sent to the first electronic device 512. In this example, the system further comprises augmented or mixed reality rendering apparatus at the first site 510, such as an augmented or mixed reality headset. In this way, the first engineer is able to ‘see’ the next step superimposed upon his or her reality, and so ‘see’ the next step superimposed upon the first radiotherapy device 511. Where the next step is generated as mixed reality content, the first engineer is also able to ‘explore’ and ‘interact’ with the mixed reality content. The augmented or mixed reality content may be representative of the or a part of the first radiotherapy device 511: for example, the augmented or mixed reality content may be representative of a correct orientation of a part of the first radiotherapy device 511. Alternatively, or additionally, the augmented or mixed reality content may contain data which describes the solution or next steps: for example, the augmented or mixed reality content may contain a list of next steps. When the augmented or mixed reality content contains both a representation of the radiotherapy device 511 and data describing the solution or next steps, the solution or next steps are rendered beside or are overlaid on top of the representation of the first radiotherapy device 511. As a result, when rendered, the first engineer can ‘see’ both the representation of the first radiotherapy device 511 and the solution or next steps overlaid on top of the actual first radiotherapy device 511.

In summary, advantages of this approach include that the first engineer is provided with a high level of detail on how the next step is to be carried out and so, not only is the first engineer's training improved, but the time to install or downtime of the first radiotherapy device 511 is reduced as the first engineer is able to quickly and effectively implement the next step.

### 8. Sixth Implementation—Physically Mirroring the Problem and Providing Guidance Using Augmented, Virtual or Mixed Reality

In a sixth implementation, the system further comprises a second radiotherapy device or a portion of a second radiotherapy device located at the second site 520. In the sixth implementation, the second engineer physically recreates the issue on the second radiotherapy device or the portion of the second radiotherapy device.

In an example of the sixth implementation, the first 511 and second radiotherapy devices are substantially the same. Alternatively, the first 511 and second radiotherapy device are not substantially the same. Conversely, the first and second radiotherapy devices are similar to the extent that the issue may be recreated on the second radiotherapy device. The second radiotherapy device may not be an ‘active’ device—that is, it may be a model device, rather than a working or workable device.

In a further example of the sixth implementation, where the first engineer is installing the first radiotherapy device 511, on receiving data identifying an issue with the installation of the first radiotherapy device 511, the second engineer strips the second radiotherapy device, or the portion of the second radiotherapy device, back to a same stage of installation. Where there is a specific issue, the second engineer recreates the issue on the second radiotherapy device. The next step of installation is then performed on the second radiotherapy device. The performance of the next step is captured by video or image, for example. The data identifying the next step is then sent to the first electronic device 512 and may be displayed to the first engineer in a way previously described. In this way, the second engineer is able to provide detailed guidance to the first engineer, which improves the likelihood of the second engineer successfully carrying the next step out on the first radiotherapy device 511, thus reducing the installation time, or downtime.

Where the first engineer is maintaining the first radiotherapy device 511, on receiving data identifying an issue with the maintenance of the first radiotherapy device 511, the second engineer modifies the second radiotherapy device in such a way that the second radiotherapy device exhibits the issue. The next step may then be performed, captured, and sent to the first electronic device 512 in the way described above.

In another example, augmented reality content, virtual reality content or mixed reality content is generated illustrating the next step being performed on the second radiotherapy device. Such content may then be sent to the first electronic device 512 and implemented as discussed above.

### 9. System Feature: Solution Database

In an example, the system further comprises a solution database. The data which forms the solution database may be queried by the second data controller and/or the first data controller. The solution database may store data identifying each issue reported to the second site 520 from the first site 510, along with data identifying the next step for overcoming the issue.

Advantageously, this means that when data is received at the second electronic device 522 identifying an issue with the first radiotherapy device 511, data identifying the next step need not necessarily be generated. Instead, data identifying the next step for overcoming the issue may be searched for in the solution database. If a result is returned, the result—which identifies the next step for overcoming the issue—is sent to the first electronic device 512. As a result, it is possible to very quickly provide the first electronic device 512 with data identifying the next step as the data need not be generated from scratch. The first engineer may therefore quickly implement the next step and so installation time and downtime are reduced.

The solution database may be stored on a central memory. The central memory may comprise a server, or a number of different servers, as part of a cloud storage solution. The central memory may be accessible by the first and/or second electronic devices.

In an arrangement, the central memory is accessible by the first electronic device 512 and the first electronic device 512 is configured to query the solution database for data identifying a next step for overcoming an issue identified with the first radiotherapy device 511. If a result is returned, the result—which identifies the next step for overcoming the issue—is sent to the first electronic device 512. In this way, the first electronic device 512 may quickly look up and return the next step for overcoming the issue with the first radiotherapy device 511 without engaging the second electronic device 522 (or second engineer). If a result is not returned—that is, a pre-existing next step does not already exist in the solution database—the first electronic device 512 sends data identifying the issue to the second electronic device 522 in the way previously described. In some arrangements, a local copy of the solution database may be stored in a memory of the first electronic device 512.

Additionally, or alternatively, the solution database may comprise a solution protocol. That is, the solution database may comprise a solution protocol and/or data identifying each issue reported to the second site 520 from the first site 510, along with data identifying the next step for overcoming the issue.

The solution protocol may comprise guidelines which engineers, such a first engineer, can refer to and apply. In particular the guidelines may comprise sets of instructions which an engineer, such as first engineer, may follow to investigate and address a particular issue with the first radiotherapy device 511. The guidelines, and sets of instructions, may take the form of any of the media formats described in this disclosure: for example, audio, video, augmented reality, and so on. The guidelines may be substantially the same as the previously described data identifying the next step for overcoming the issue

The guidelines may be updated. For example, the guidelines may be updated to reflect that one or more of the following has occurred:
Updates have been applied to the first radiotherapy device 511 resulting in it being necessary to update the guidelines.
New guidelines have been issued. New guidelines may be created and issued in reaction to an issue seen or reported in real world radiotherapy devices. For example, if a same issue is reported several times, a specific set of guidelines for handling that issue may be generated. Such reactive creation and issuance of new guidelines may be based on the data identifying issues reported to the second site 520 from the first site 510. Alternatively, or additionally, new guidelines may be created and issued based on predicted future failures of the radiotherapy machines.

The guidelines may include information on the replacement and/or calibration of parts, for example.

A key advantage of the solution database is that it is a ‘one stop shop’ for solutions for radiotherapy device issues. As a result of compiling all the solutions in a single place, in addition to other technical advantages, power usage and processing power are all reduced.

This is primarily because the solution or guidance need only be generated once per issue, rather than each time the issue occurs.

### 10. System Feature: Tiered Solution System

In an example, the solution database is a tiered system. In more detail, the information stored in the solution database is labelled according to a tiered system. For example, the tiered system may comprise: tier 1; tier 2; and tier 3. Tier 1 may be used to indicate information which is accessible and/or implementable by a non-expert e.g. a hospital technician. In this way, tier 1 may indicate to a non-expert that he or she may implement the corresponding solution, guidance or next step defined in the solution database. For example, a tier 1 solution may include guidance on replacing a fuse in the radiotherapy device. Meanwhile, tier 2 may be used to indicate information which is accessible and/or implementable by a part-expert e.g. a hospital technician with radiotherapy device training. In this way, tier 2 may indicate to a part-expert that he or she may implement the corresponding solution, guidance or next step defined in the solution database; however, a non-expert may not. Finally, tier 3 may be used to indicate information which is accessible and/or implementable by an expert e.g. an engineer who is specifically trained in the maintenance of the radiotherapy device(s) in question. In this way, tier 3 may indicate to an expert that he or she may implement the corresponding solution, guidance or next step defined in the solution database; however, a non-expert or part-expert may not.

The specific tiered system described here is intended as an example only. The key principle is that each solution defined in the solution database may be labelled to indicate a required experience level for accessing and/or implementing the respective solution. The require experience level may differ geographically, for example according to local laws and needs.

### 11. System Feature: Logging Reported Issues

In an example, each time data identifying an issue is received at the second electronic device 522 and/or or each time the solution database (where present) is queried for a next step for an issue (by either the first electronic device 512 or the second electronic device 522), a notification is sent to the second electronic device 522. The notification identifies at least the issue, and—in some examples—the notification additionally identifies the radiotherapy device about which the issue is related.

The second data controller processes the notifications received at the second electronic device 522 and creates a log of issues reported to the second electronic device 522 and/or queried in the solution database. The log may be saved in the central memory. The log may comprise: data identifying the issues; the number of times a notification corresponding to each issue has been received; the frequency with which notifications corresponding to each issue have been received; and/or the issues received in connection with each radiotherapy device deployed at a user location.

Advantageously, the log can be used to better understand the health of the deployed radioactive devices. This information can be used to inform research and development and training. For example, where a particular issue is happening frequently with a certain component, research into how the issue might be avoided can be made a research and development priority and/or, in the meantime, training can be given on how to resolve the issue such that field-service engineers have the know-how to resolve the problem. As a result, the radiotherapy devices themselves may be made more resilient and, in the meantime, installation and maintenance may be carried out more efficiently.

### 12. System Feature: Security

In an example, at least some of the data sent between the first 512 and second 522 electronic devices is encrypted. For example, the data may be encrypted using a symmetric-key scheme (i.e. the encryption and decryption keys are the same), or a public-key encryption scheme (i.e. the encryption key is made public, however the decryption key is private). In an example, the data identifying the issue is not encrypted, however the data identifying the next step for addressing the issue is encrypted. Usefully, this means that third parties would not be able to intercept and read the data identifying the next step for addressing the issue. This avoids third parties attempting to implement the next step themselves, which could be highly dangerous.

In a further example, data stored semi-permanently or permanently on any of the first 512 or second 522 electronic devices or in the central memory has security provisions applied. Security provisions may comprise encrypting the data or applying password protection, for example.

### 13. System Feature: Site Locations and Connectivity

As set out above, the first 510 and second 520 sites of the system shown in FIG. 3 are remote from each other. The term ‘remote’ may mean arranged such that persons at the first 510 and second 520 site cannot communicate directly with each other. Instead, a communication channel such as Wi-Fi must be used, for example.

Accordingly, in this disclosure, first 510 and second 520 sites housed in respective buildings may be considered as being remote from each other, regardless of the actual proximity of the first 510 and second 520 site to each other.

In an example, in order to communicate directly and securely between remote sites, the first engineer, for example, may carry with him or her a WiFi router. The WiFi router is configured to generate a safe and secure WiFi network when plugged into an NSS Service Port. In use, on arrival at a remote site, such as the first site 510, the first engineer may locate an NSS Service Port and plug the WiFi router into the NSS Service Port in order to establish a WiFi network. The first engineer may then communicate with a remote site, such as the second site 520, over the established WiFi network. Advantageously, this provides a secure and reliable communication channel between first and second sites 510, 522, which are remote from each other. A further advantage of this approach is that variations in internet connectivity between different sites, particularly between different sites around Europe, is accounted for and addressed as the first engineer uses the WiFi router to establish a dedicated, safe and secure WiFi network.

As the skilled person will well appreciate, radiotherapy devices, such as the first radiotherapy device 511, are typically provided in so called bunkers. Bunkers are designed to contain radiation originating from the radiotherapy device and so prevent unintentionally exposing people or animals to radiation. NSS Service Ports such as those described in the preceding paragraph may be provided inside such bunkers. Advantageously this means that the first engineer may communicate with the second site 520 whilst in the presence of the radiotherapy device being examined—that is, whilst inside the bunker.

In other examples, a WiFi network may already be available in the bunker. For example, where the bunker is provided in a hospital, there may be a hospital WiFi network available for use in the bunker which the first engineer may connect to.

In other examples, a cellular network may be available in the bunker and the devices used by the first engineer may be connectable to the cellular network. For example, there may be 3G, 4G and/or 5G networks available in the bunker.

In other examples, the bunker may be provided with a hard wired internet connection, such as an ethernet cable connection. Accordingly, the device(s) used by the first engineer may be connectable with such hard wired internet connections.

In some examples, tunneling is used to communicate between the first 510 and second 520 sites. Tunneling is an internet protocol used to allow private network communications to be sent across a public network, such as the internet. Advantageously, the use of tunneling improves the security of the communication link between the first 510 and second 520 sites since the information exchanged via tunneling is kept private.

### 14. System Feature: Data Indicative of an Issue with the Radiotherapy Device

Data identifying an issue with the first radiotherapy device 511 may comprise data identifying a current status of the radiotherapy device. That is, the data identifying an issue with the first radiotherapy device 511 need not be data identifying a problem with the radiotherapy device. Instead, the first engineer may simply reach a stage of installation or maintenance and be unsure as to the best next step, for example. In such an example, the data identifying an issue with the first radiotherapy device 511 comprises data identifying the stage of installation or maintenance reached and a request for guidance on the next step.

### 15. System Feature: Software Issues

The described approach to identifying and addressing issues with radiotherapy devices need not be limited to hardware related issues. Instead, the described approach may additionally or alternatively apply equally well to software issues with radiotherapy devices, or software products usable and/or complementary to the use of such radiotherapy devices.

### 16. Combinations of Implementations and System Features

In ways which will be apparent to the skilled person, elements of various ones of the implementations and system features described above may be combined. Indeed, such combinations are envisaged and form part of this disclosure. In particular, it is envisaged that various ones of the described data types (such as audio, image, video, environment, and so on) may be combined.

Further, although specific reference has been made in this disclosure to linac machines, the described approach may equally well apply to both hardware and software other than linac machines. For example, the described approach may equally well apply to other types of radiotherapy machine. Further, the described approach may equally well apply to providing software solutions, as has been described.

### 17. Method—Reporting the Issue

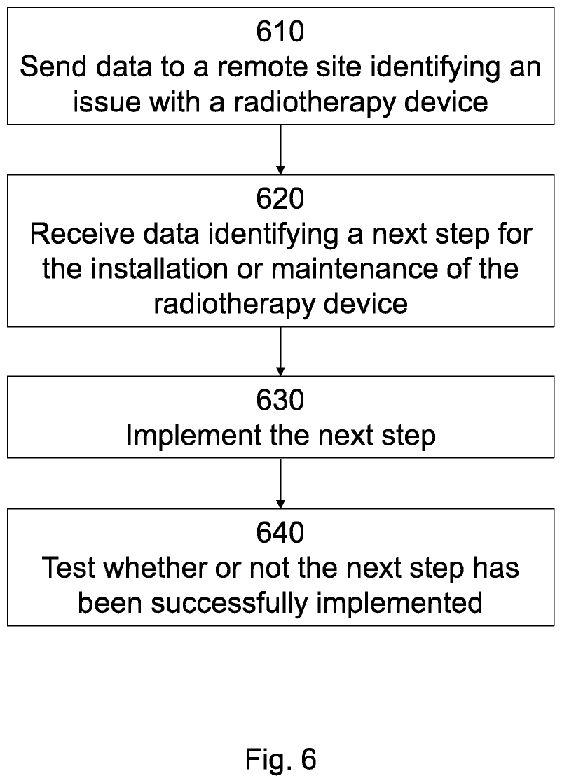

FIG. 6 shows a method according to the present disclosure. The method is suitable for being carried out by the first electronic device 512 at the first site. At step 610, data identifying an issue with the installation or maintenance of a radiotherapy device is sent to a remote site, such as the second site 520. The data identifying the issue may be sent from the first electronic device 522. The data may be sent using any of the communication methods previously discussed, and in any of the data forms previously discussed. Where the issue relates to the installation of the radiotherapy device, the data identifying the issue may comprise information sufficient for the recipient of the data to ascertain a current stage of installation of the radiotherapy device.

At step 620, data is received identifying the next step for the installation or maintenance of the radiotherapy device. The data identifying the next step may be sent from the second electronic device 522 and may be sent using any of the communication methods previously discussed and in any of the forms previously discussed. The data identifying the next step may be received at the first electronic device 512. The data identifying the next step may be stored in a memory. For example, the data identifying the next step may be stored in a memory of the first electronic device 512. The data identifying the next step comprises information sufficient for the recipient of the data to install or maintain—at least in part—the first radiotherapy device. For example, the data identifying the next step may comprise an instruction to move a particular component of the radiotherapy device to a different position, or to connect multiple components of the radiotherapy device together.

At step 630, the next step for the installation or maintenance of the radiotherapy device is implemented. The next step may be implemented by a field-service engineer in attendance at the location of the radiotherapy device. Alternatively, the next step may be implemented by a robot under control of a computer system, such as by use of a robotic arm.

At step 640, a test may be carried out to determine whether or not the next step has been successfully implemented. The test may comprise determining whether the issue has been resolved. Determining whether the issue has been resolved may be carried out using diagnostic software. The test need not be carried out directly after execution of the next step; instead, the test may be carried out after subsequent installation and/or maintenance steps.

The test may additionally or alternatively comprise returning to step 610 of the method. In this case, the data identifying an issue with the radiotherapy device comprises data identifying a current status of the radiotherapy device. In this example, if the next step has been successfully implemented, the data identifying the next step may comprise an instruction to move on to the next stage of installation or maintenance and/or an indication that the next step was successfully implemented and/or an indication that installation or maintenance of the radiotherapy device is complete. Thus, the test may include ‘outsourcing’ the question of whether the next step has been successfully implemented.

### 18. Method—Generating the Solution

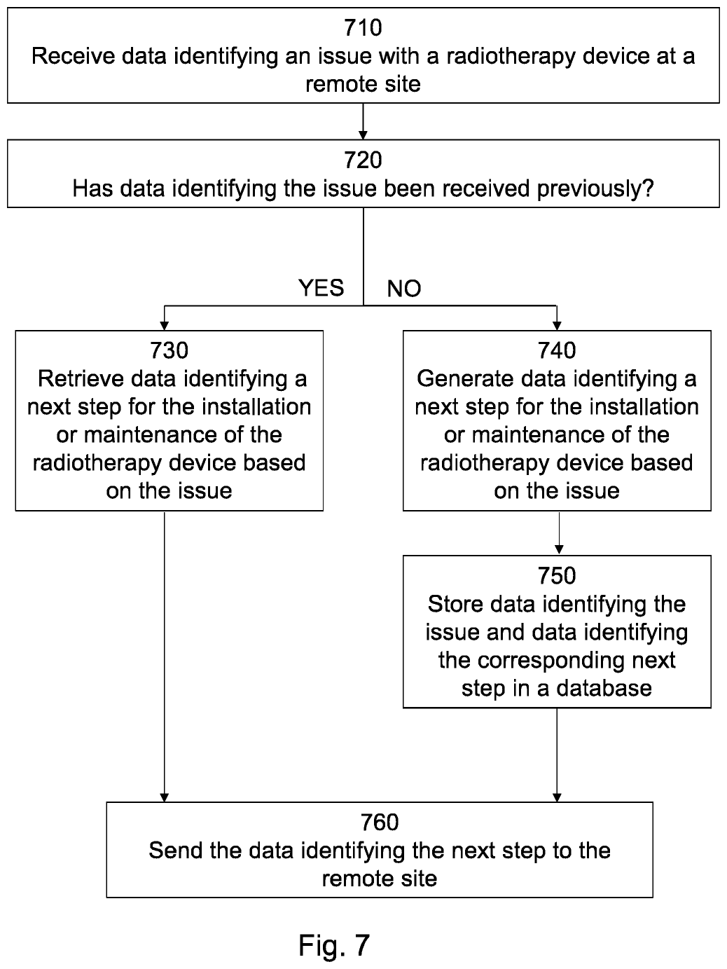

FIG. 7 shows a method in accordance with the present disclosure. The method is suitable to be carried out, for example, by the second electronic device 522.

At step 710, data identifying an issue with a radiotherapy device at a remote site is received. The data may be sent from the first electronic device 512, for example. The data may be received at the second electronic device 522. The data identifying the issue may be sent using any of the communication methods previously discussed and in any of the data forms previously discussed. The data may be stored in a memory. For example, the data may be stored in a memory of the second electronic device 522.

At step 720, it may be determined whether or not data has been received previously identifying the issue. This determination may be carried out by the second data controller and/or may be carried out by querying the solution database previously described. If data identifying the issue has been received previously—for example, the result at step 720 is ‘yes’—step 730 is carried out. If data identifying the issue has not been received previously—for example, the result at step 720 is ‘no’—step 740 is carried out. Each of steps 730 and 740 is described in detail below.

At step 730, data is retrieved which identifies the next step for the installation or maintenance of the radiotherapy device based on the issue (that is, based on the data identifying the issue received at step 510). The data identifying the next step may be stored in, and retrieved from, the previously described solution database.

Advantageously, in the case where step 730 is executed—that is, in the case where data identifying the issue has been received previously—data identifying the next step is provided to the remote site very quickly. This means that the next step can be implemented very quickly, reducing downtime or installation time. Furthermore, steps 720 and 730 may be automated, for example steps 720 and 730 may be executed under control of the second electronic device 522. In such an example, an efficient technical solution for guiding field-service engineers on the appropriate next installation or maintenance step is provided.

Alternatively, moving instead to step 740, at step 740 data is generated identifying a next step for the installation or maintenance of the radiotherapy device based on the issue. The data identifying the next step may be generated and may be in any of the forms previously described.

At step 750, the data identifying the issue and the data identifying the corresponding next step for the installation or maintenance of the radiotherapy device may be saved in a database, such as the solution database. As a result, if data is subsequently received identifying the same issue, the outcome at step 720 will be ‘yes’ and step 730 may be executed, rather than step 740.

At step 760, the retrieved data (in the case of step 730 being executed) or the generated data (in the case of step 740 being executed) identifying the next step for the installation or maintenance of the radiotherapy device is sent to the remote site where the radiotherapy device 511 is located.

### 19. Examples of Centralising the Competency

According to a first example, there is provided a method for identifying a next step for installing or maintaining a radiotherapy device, the radiotherapy device comprising a linear accelerator and being configured to provide therapeutic radiation to a patient; the method comprising: sending, to a remote site, data identifying an issue with the radiotherapy device; and receiving, from the remote site, data identifying a next step for installing or maintaining the radiotherapy device, the data identifying the next step being based upon the data identifying the issue.

The method may be a method for identifying a next step for installing or maintaining software associated with the radiotherapy device. The data identifying the issue with the radiotherapy device may comprise data identifying the issue with the software associated with the radiotherapy device. Data identifying the next step for installing or maintaining the radiotherapy device may comprise data identifying the next step for installing or maintaining the software associated with the radiotherapy device.

The method may further comprise the step of: installing or maintaining the radiotherapy device according to the data identifying the next step.

The data identifying the issue with the radiotherapy device may comprise one or more of: audio data; video data; image data; augmented reality data; and mixed reality data. The data identifying a next step for installing or maintaining the radiotherapy device may comprise one or more of: audio data; video data; image data; augmented reality data; mixed reality data; and virtual reality data.

The method may further comprise the step of rendering the data identifying the next step for installing or maintaining the radiotherapy device on a first electronic device.

The method may further comprise the step of rendering the data identifying the issue with the radiotherapy device on a second electronic device.

The first electronic device may comprise one or more of: a laptop computer; a tablet computer; a smartphone; and an augmented reality or mixed reality wearable.

The second electronic device may comprise one or more of a laptop computer; a tablet computer; a smartphone; and an augmented reality or mixed reality wearable.

The data identifying the next step for installing or maintaining the radiotherapy device may be generated by physically mirroring the issue. The issue may be mirrored on at least a portion of a further radiotherapy device. The further radiotherapy device may be a model device. The data identifying the next step for installing or maintaining the radiotherapy device may be generated by virtually mirroring the issue. The issue may be mirrored using computer-aided design software.

When the data identifying the issue with the radiotherapy device comprises augmented reality data and/or mixed reality data, the method may further comprise generating the augmented reality data and/or mixed reality data using a scanner. The scanner may be communicatively coupled to a data controller. The data controller may be configured to receive scanner data from the scanner; process the scanner data; and generate, from the processed scanner data, a digital three-dimensional model of the environment.

The method may further comprise storing, in a solution database, the data identifying the issue with the radiotherapy device and/or the data identifying the next step for installing or maintaining the radiotherapy device.

The solution database may further comprise a solution protocol. The solution protocol may comprise guidelines for investigating and addressing one or more issues with the first radiotherapy device. The guidelines may be based at least in part on the data identifying the issue with the radiotherapy device.

The information stored in the solution database may be labelled according to a tiered system. Each tier in the system may indicate an access and/or implementation requirement for information labelled with the respective tier.

According to a second example, there is provided a system comprising a radiotherapy device, a processor, a computer readable medium, a transmitter, and a receiver; the radiotherapy device comprising a linear accelerator and being configured to provide therapeutic radiation to a patient; wherein the computer readable medium comprises computer executable instructions which, when executed by the processor, cause the processor to: send, to a remote site via the transmitter, data identifying an issue with the radiotherapy device; and in response to receiving, via the receiver, data from the remote site identifying a next step for installing or maintaining the radiotherapy device, providing an indication of the next step; wherein the data identifying the next step is based upon the data identifying the issue.

According to a third example, there is provided a method for identifying, from a remote site, a next step for installing or maintaining a radiotherapy device, the radiotherapy device comprising a linear accelerator and being configured to provide therapeutic radiation to a patient; the method comprising: receiving, at a remote site, data identifying an issue with the radiotherapy device; and sending, from the remote site, data identifying a next step for installing or maintaining the radiotherapy device, the data identifying the next step being based upon the data identifying the issue.

The method may further comprise the step of generating the data identifying the next step based upon the data identifying the issue.

The remote site may be a physical location remote from the physical location of the radiotherapy device.

According to a fourth example, there is provided a system at a remote site comprising a processor, a computer readable medium, a transmitter, and a receiver; wherein the computer readable medium comprises computer executable instructions which, when executed by the processor, cause the processor to: in response to receiving, via the receiver, data identifying an issue with the radiotherapy device, the radiotherapy device comprising a linear accelerator and being configured to provide therapeutic radiation to a patient, identify a next step for installing or maintaining the radiotherapy device; and send, via the transmitter, data identifying the next step for installing or maintaining the radiotherapy device, wherein the data identifying the next step is based upon the data identifying the issue.

The data identifying an issue with the radiotherapy device may be received from a physical location associated with the radiotherapy device. The data identifying the next step for installing or maintaining the radiotherapy device may be sent to the or a physical location associated with the radiotherapy device. The radiotherapy device may be at the physical location.

According to a fifth example, there is provided a method for identifying a next step for installing or maintaining a radiotherapy device at a first site, the radiotherapy device comprising a linear accelerator and being configured to provide therapeutic radiation to a patient; the method comprising: sending, from the first site, data identifying an issue with the radiotherapy device; receiving, at the remote site, data identifying an issue with the radiotherapy device; identifying, at the remote site, a next step for installing or maintaining the radiotherapy device; sending, from the remote site, data identifying a next step for installing or maintaining the radiotherapy device, the data identifying the next step being based upon the data identifying the issue; and receiving, at the first site, the data identifying the next step.

Optional features of examples may apply equally well to others of the examples.

The above implementations have been described by way of example only, and the described implementations and arrangements are to be considered in all respects only as illustrative and not restrictive. It will be appreciated that variations of the described implementations and arrangements may be made without departing from the scope of the invention and that such variations are envisaged and intended to be within the scope of the present disclosure.

1. A method for identifying an issue with a radiotherapy device and outputting a next step, the radiotherapy device comprising a linear accelerator and being configured to provide therapeutic radiation to a patient, the method comprising:
obtaining data relating to an operational state of at least one of the radiotherapy device or a component thereof identifying an issue with the radiotherapy device based on the obtained data; and
outputting the next step based on the identified issue.

2. The method according to claim 1, wherein the data related to the operational state is obtained by performing a diagnostic routine on at least one of the radiotherapy device or the component thereof.

3. The method according to claim 2, wherein the diagnostic routine is a remote diagnostic routine controlled from a remote location or is a self-diagnostic routine performed by the radiotherapy device, or wherein the diagnostic routine is a predictive maintenance routine.

4. The method according to claim 2, wherein the diagnostic routine comprises:
receiving data from the radiotherapy device indicative of performance of one or more components of the radiotherapy device, and
outputting the diagnostic routine based on a comparison of the data indicative of the performance of the one or more components with threshold data.

5. The method according to claim 1, further comprising:
assigning a category to the issue.

6. The method according to claim 5, wherein the category is indicative of a severity of the issue.

7. The method according to claim 5, wherein assigning the category comprises calculating a linac health score (LHS) for the radiotherapy device using:
$$L H S = ( λ \cdot {\prod }_{i} \frac{R U {L}^{i}}{E U {L}^{i}} \cdot {w}^{i} + ( 1 - λ ) \cdot \frac{total expected alarms scores}{weighted sum of alarms scores} ) \times 100$$
wherein each critical part i, has a prediction of a residual useful life (RUL), a total expected useful life (EUL), and a corresponding weight (ω) in a total machine health score based on risk contribution of each critical part.

8. The method according to claim 5, wherein, prior to outputting the next step, the next step is determined based on the category.

9. The method according to claim 1, wherein the next step comprises fully or partially disabling operation of the radiotherapy device.

10. The method according to claim 1, wherein the next step comprises limiting or changing a functionality of the radiotherapy device.

11. The method according to claim 1, wherein the next step further comprises:
changing patient scheduling by rescheduling a treatment date or time for the patient.

12. The method according to claim 1, wherein the next step comprises:
changing patient scheduling by changing al associated radiotherapy machine for a particular patient treatment.

13. The method according to claim 1, wherein the next step comprises:
requesting an authorized engineer attend to the radiotherapy device.

14. The method according to claim 1, wherein the next step comprises:
requesting at least one of a device specialist or an onsite technician attend to the radiotherapy device.

15. The method according to claim 1, wherein the next step comprises:
recording the issue in a database.

16. The method according to claim 15, further comprising:
resolving, at a next scheduled service of the radiotherapy device, the issue.

17. The method according to claim 16, wherein the issue is resolved by implementing a maintenance or installation step at the radiotherapy device.

18. The method according to claim 1, wherein the next step comprises:
determining a series of steps for overcoming the issue.

19. The method according to claim 18, further comprising:
displaying the series of steps, in sequence, on at least one of a screen of a user device or a screen of the radiotherapy device.

20. The method according to claim 19, wherein displaying the series of steps, in sequence comprises, for each next step:
displaying a preceding step in the sequence;
receiving confirmation from a sensor associated with the radiotherapy device that the preceding step in the sequence has been successfully implemented; and
displaying a subsequent step in the sequence.

21. The method according to claim 19, comprising:
displaying the next step or the series of steps via an augmented reality rendering apparatus.

22. The method according to claim 1, further comprising:
prior to outputting the next step based on the identified issue, identifying the next step.

23. The method according to claim 22, wherein identifying the next step comprises:
sending, to a remote site, data identifying the issue with the radiotherapy device; and
receiving, from the remote site, data identifying the next step for installing or maintaining the radiotherapy device, the data identifying the next step being based upon the data identifying the issue.

24. A non-transitory computer readable medium comprising instructions which, when performed by a processor, cause the processor to:
obtain data related to an operational state of at least one of a radiotherapy device or a component of the radiotherapy device;
identify an issue with at least one of the radiotherapy device or the component of the radiotherapy device from the data; and
output a next set based on the identified issue.

25. The non-transitory computer readable medium of claim 24, comprising instructions to be executed by a plurality of processors arranged at a plurality of different physical locations.

26. A system comprising:
a radiotherapy device comprising a linear accelerator and being configured to provide therapeutic radiation to a patient;
a processor;
a transmitter;
a receiver; and
memory, with instructions stored thereon that, when executed by the processor, cause the processor to:
obtain data related to an operational state of at least one of a radiotherapy device or a component of the radiotherapy device;
identify an issue with at least one of the radiotherapy device or the component of the radiotherapy device from the data; and
output a next set based on the identified issue.

27. The system of claim 26, further comprising
one or more sensors associated with the radiotherapy device, wherein each sensor is arranged to collect the data relating to the operational state of the radiotherapy device or the component of the radiotherapy device.

28. A method for limiting or changing a radiotherapy device functionality, the method comprising:
obtaining data relating to an operational state of the radiotherapy device or a component thereof;
identifying an issue with the radiotherapy device based on the obtained data; and
limiting or changing the radiotherapy device functionality based on the identified issue.
